(19) United States
(12) Patent Application Publication
(10) Pub. No.: US 2024/0408019 A1
(43) Pub. Date: Dec. 12, 2024

(54) METHODS FOR FREEZING AND FREEZE-DRYING LIPID NANOPARTICLES (LNPs) AND LNPs OBTAINED WITH THE SAME

(71) Applicant: SANOFI, Paris (FR)

(72) Inventors: Etienne BOUTRY, Paris (FR); Florent PERAL, Paris (FR); Bertrand WOINET, Paris (FR)

(21) Appl. No.: 18/698,580

(22) PCT Filed: Oct. 4, 2022
(86) PCT No.: PCT/EP2022/077571

§ 371 Date: Apr. 4, 2024

(30) Foreign Application Priority Data
Oct. 5, 2021 (EP) 21306387.8

(51) Int. Cl. A61K 9/19; A61K 9/51; A61K 31/7105

(52) U.S. Cl. CPC A61K 9/19; A61K 9/5123; A61K 31/7105

(57) ABSTRACT

The present invention relates to a method for freezing or freeze-drying lipid nanoparticles (LNPs) comprising at least a nucleic acid and, at least, as lipid components, a cationic ionizable lipid, a neutral lipid, and a steroid alcohol, or an ester thereof. The method comprises the steps of providing a liquid composition comprising said LNPs, spraying the composition of step a) in conditions suitable for obtaining liquid droplets, and freezing the liquid droplets obtained at step b) to obtain frozen LNPs. The method may also comprise a step of drying the frozen LNPs obtained to obtain freeze-dried LNPs. The invention also relates to frozen and freeze-dried LNPs.

### TECHNICAL FIELD

The present invention relates to field of pharmaceutical formulations and methods of preparing thereof. The present invention further relates to methods for freezing and freeze-drying lipid nanoparticles (LNPs).

### TECHNICAL BACKGROUND

Lipid nanoparticles (LNPs) have proven efficient to deliver various types of therapeutic active agents into cells (Thi et al., Vaccines, 2021, 9(4), 359). For example, LNPs containing nucleic acids, such as LNP-mRNA, have attracted a great interest and have recently proven their efficacy and safety in vaccine fields and have proven dramatically important in the management of the Covid-19 pandemic (Reichmuth et al., Therapeutic delivery, 2016, 7(5), 319-334; Khurana et al., Nano today, 2021, 38, 101142).

However, an important drawback of the currently licensed mRNA-lipid nanoparticles (LNPs) COVID-19 vaccines is that they have to be stored at very low 20 temperatures (Schoenmaker et al., International journal of pharmaceutics, 2021, 601, 120586). Indeed, the storage temperature conditions required for the both licensed vaccines are respectively of about −20° C. for the Moderna's vaccine and about −80° C. to −60° C. for the BioNTech/Pfizer's vaccine (Crommelin et al., J Pharm Sci. 2021; 110(3):997-1001).

LNPs which are organized structures may be negatively impacted by the freezing process and such storage temperatures. Alterations in LNPs structure may result in a reduced capacity of the LNPs to properly deliver their cargo to the cells and to achieve the intended therapeutic effect.

Furthermore, such temperature conditions necessarily create multiple difficulties in the maintenance of the cold chain all through the manufacturing, distribution, and packaging of the pharmaceutical compositions. Any failure in the cold chain management will necessarily end up in product waste.

While a pandemic situation requires a prompt and global answer, the demands in temperature level and control required to ensure the maintenance of LNPs-based vaccines qualities may slow the deployment of manufacturing facilities, global distribution organizations, and local supplies and administration.

Freeze-drying (or lyophilization) is a common method to stabilize labile product in pharmaceutical industry. Freeze-drying is a technique by which a product is solidified by freezing and the solvent that contains it, such as water, is evaporated by sublimation upon heating under low atmospheric pressure (or vacuo) or under a flow of cold, dry, gas. Freeze-drying may be carried by conventional lyophilization in vials or by spray-freeze drying (SFD).

Methods and apparatus for spray-freeze drying are disclosed in Adali et al., Processes. 2020; 8(6), Wanning et al., Int J Pharm. 2015; 488(1-2):136-153, WO 2009/109550 A1, WO 2013/050162 A1, WO 2013/050156 A1, or WO 2013/050159 A1.

Ali et al., (Int J Pharm. 2017; 516(1-2):170-177) describe the spray-freeze-drying of lipid nanoparticles.

Fukushige et al., (Int J Pharm. 2020; 583:119338) describe the spray-freeze-drying of liposomes containing protamine-siRNA complexes.

Zhao et al., Bioact Mater. 2020; 5(2):358-363 report a study on the stability of lipid-like nanoparticles (LLNs) containing mRNA with different concentrations of cryoprotectants (sucrose, trehalose or mannitol) under the conditions of freezing or lyophilization processes.

However, freeze-drying of LNPs is also known to cause several stresses leading to physical instability, e.g. aggregation, fusion, or content leakage, of the LNPs (Trenkenschuh et al., Eur J Pharm Biopharm. 2021 August; 165:345-360). Ball et al. (Int J Nanomedicine, 2016, 12, 305-315) report the impact of lyophilization on LNPs stability.

Therefore, there is still a need for methods for freezing or freeze-drying LNPs having no or reduced negative impact on LNPs structure and/or stability.

There is a need for methods for freezing or freeze-drying LNPs having no or reduced effect on LNPs aggregation and sizes distribution of the LNPs.

There is a need for methods for freezing or freeze-drying LNPs having no or reduced effect on encapsulation rate of agents encapsulated in LNPs.

There is a need for LNPs formulations suitable for being spray-frozen or spray-freeze-dried with no or reduced negative impact on LNPs structure and/or stability.

There is a need for LNPs formulations suitable for being spray-frozen or spray-freeze-dried with no or reduced effect on LNPs aggregation and sizes distribution of the LNPs.

There is a need for LNPs formulations suitable for being spray-frozen or spray-freeze-dried with no or reduced effect on encapsulation rate of agents encapsulated in LNPs.

There is a need for methods for freeze-drying LNPs and/or for LNPs formulations suitable for being freeze-dried allowing for obtaining freeze-dried LNPs able to be stored at 2-8° C., with no or reduced effect on LNPs aggregation and sizes distribution of the LNPs.

There is a need for methods for freeze-drying LNPs and/or for LNPs formulations suitable for being freeze-dried allowing for obtaining freeze-dried LNPs able to be stored at 2-8° C., with no or reduced effect on encapsulation rate of agents encapsulated in the LNPs, such as mRNA.

There is a need for methods for freeze-drying LNPs containing mRNA and/or for LNPs formulations containing mRNA suitable for being freeze-dried, suitable for providing freeze-dried LNPs able to be stored at 2-8° C., with no or reduced effect on encapsulation rate of encapsulated mRNA.

There is a need for methods for freeze-drying LNPs containing mRNA and/or for LNPs formulations containing mRNA suitable for being freeze-dried, suitable for providing freeze-dried LNPs able to maintain or to produced enhanced protein expression from the mRNA after administration.

The present invention has for purpose to meet all or part of those needs.

### SUMMARY

According to one of its objects, the present invention relates to a method for freezing lipid nanoparticles (LNPs), said LNPs comprising at least, as lipid components, a cationic ionizable lipid, a neutral lipid, and a steroid alcohol, or an ester thereof, said LNPs comprising at least one nucleic acid, wherein said method comprises the steps of:
a) providing a liquid composition comprising said LNPs,
b) spraying the composition of step a) in conditions suitable for obtaining liquid droplets, and
c) freezing the liquid droplets obtained at step b) to obtain frozen LNPs.

As shown in the Examples section, the inventors have surprisingly observed that the freezing of liquid droplets containing LNPs, such as LNPs comprising at least, as lipid components, a cationic ionizable lipid, a neutral lipid, and a steroid alcohol, or an ester thereof, and optionally a PEG-lipid, at a freezing temperature of for example −80° C., allowed reducing aggregation of the LNPs compared to the freezing in vials. The sizes distribution of the LNPs was maintained. Further, for LNPs containing a nucleic acid, such as a mRNA, the nucleic acid encapsulation rate was observed as being higher for LNPs frozen by in droplets compared to LNPs frozen in vials. A method as disclosed herein allows advantageously for maintaining the stability of frozen LNPs.

Reduction of LNPs aggregation can be beneficial at the time of injection of formulations reconstituted from frozen LNPs as aggregates constituting large lumps may cause adverse reactions such as pain. Further, the maintenance of a good nucleic acid, such as mRNA, encapsulation rate will improve the corresponding protein expression and therefore the therapeutic intended effect.

In some embodiments, the frozen LNPs may be obtained in frozen micropellets.

According to one of its objects, the present invention relates to a method for freeze-drying lipid nanoparticles (LNPs), said method comprises at least the steps of:
d) obtaining frozen LNPs according to the method as disclosed herein, and
e) drying the frozen LNPs obtained at step d) under conditions suitable to obtain freeze-dried LNPs.

As shown in the Examples section, the inventors have surprisingly observed that the spray-freeze drying of LNPs, such as LNPs comprising at least, as lipid components, a cationic ionizable lipid, a neutral lipid, and a steroid alcohol, or an ester thereof, and optionally a PEG-lipid, allowed preventing or reducing LNPs aggregation. Further, for LNPs containing a nucleic acid, such as mRNA, the nucleic acid encapsulation rate was observed as being maintained over time compared to conventional lyophilization process (in vials). A method as disclosed herein allows advantageously for maintaining the stability of freeze-dried LNPs.

Further, as shown in the Examples section, the inventors have surprisingly observed that injections in mice of resuspended LNPS from spray-freeze dried LNPs containing a nucleic acid, such as mRNA, encoding for a protein, were resulting in higher protein expression as compared with resuspended LNPS from conventionally lyophilized LNPs.

Reduction of LNPs aggregation can be beneficial at the time of injection of formulations reconstituted from freeze-dried LNPs as aggregates constituting large lumps may cause adverse reactions such as pain. Further, the maintenance of a good nucleic acid, such as mRNA, encapsulation rate will improve the corresponding protein expression and therefore the therapeutic intended effect.

Further, freeze-dried LNPs obtained according to the spray-freeze drying methods as disclosed herein dissolved more rapidly in water for injection or in a buffer compared to the freeze-dried LNPs obtained according to conventional lyophilization. Therefore, the spray-freeze-drying methods as disclosed herein allows advantageously to obtain freeze-dried LNPs with a reduced time for dissolution compared to conventional lyophilization.

In some embodiments, the freeze-dried LNPs may be obtained in freeze-dried micropellets.

In some embodiments, the spraying at step b) may be carried out with an electromagnetic droplets stream generator, a piezoelectric droplets stream generator, a hydraulic droplets aerosol generator, a pneumatic nozzle, a ultrasonic spray nozzle, a thermal droplets stream generator, or an electrohydrodynamic droplets (EHD) generator. In some embodiments, the spraying may be carried out with an electromagnetic droplets stream generator. In some embodiments, the spraying may be carried out with piezoelectric droplet stream generator.

The freezing of the liquid droplets may be obtained by contacting the liquid droplets with a freezing gas, a freezing liquid, or a freezing surface. In some embodiments, the step c) of freezing may be carried out by spraying the liquid droplets into a cryogenic atmosphere, with compressed carbon dioxide, into vapor over a cryogenic liquid, into a cryogenic liquid, or onto a cold solid surface. In a method as disclosed herein, the step c) of freezing may be carried out by spraying the liquid droplets into a cryogenic atmosphere.

The drying (or freeze-drying) at step e) may be carried out by rotary drum vacuum lyophilization, atmospheric drying with a flow of cold air, vacuum chamber lyophilization, or vacuum tunnel lyophilization. In some embodiments, the drying at step e) may be carried out in vacuum chamber lyophilization. In some embodiments, the drying at step e) may be carried out by rotary drum vacuum lyophilization.

In some embodiments, the LNPs may comprise:
from about 20 to about 60%, or from about 25% to about 60%, or from about 30% to about 55%, or from about 35% to about 55%, or from about 35% to about 50%, or from about 40% to about 50%, of said ionizable cationic lipid, and/or
from about 5 to about 50%, or from about 5% to about 45%, from about 9% to about 40%, from about 9% to about 30% of said neutral lipid, and/or
from about 20 to about 55%, or from about 20% to about 50%, or from about 25% to about 45%, of said steroid alcohol, or ester thereof,
in % w/w relative to the total weight of the lipid components of said LNPs.

In some embodiments,
the ionizable cationic lipid is selected from the group comprising [(6Z,9Z,28Z,31Z)-heptatriaconta-6,9,28,31-tetraen-19-yl] 4-(dimethylamino)butanoate (D-Lin-MC3-DMA); 2,2-dilinoleyl-4-dimethylaminoethyl-[1,3]-dioxolane (DLin-KC2-DMA); 1,2-dilinoleyloxy-N,N-dimethyl-3-aminopropane (DLin-DMA); di((Z)-non-2-en-1-yl) 9-((4-(dimethylamino)butanoyl)oxy)heptadecanedioate (L319); 9-heptadecanyl 8-{(2-hydroxyethyl)[6-oxo-6-(undecyloxy)hexyl]amino}octanoate (SM-102); [(4-hydroxybutyl)azanediyl]di(hexane-6,1-diyl) bis(2-hexyldecanoate) (ALC-0315); [3-(dimethylamino)-2-[(Z)-octadec-9-enoyl]oxypropyl] (Z)-octadec-9-enoate (DODAP); 2,5-bis(3-aminopropylamino)-N-[2-[di(heptadecyl)amino]-2-oxoethyl]pentanamide (DOGS); [(3S,8S,9S,1 OR,13R,14S,17R)-10,13-dimethyl-17-[(2R)-6-methylheptan-2-yl]-2,3,4,7,8,9,11,12,14,15,16,17-dodecahydro-1H-cyclopenta[a]phenanthren-3-yl] N-[2-(dimethylamino)ethyl]carbamate (DC-Chol); tetrakis(8-methylnonyl) 3,3′,3″,3′″-(((methylazanediyl) bis(propane-3,1 diyl))bis (azanetriyl))tetrapropionate (306Oi10); decyl (2-(dioctylammonio)ethyl) phosphate (9A1P9); ethyl 5,5-di((Z)-heptadec-8-en-1-yl)-1-(3-(pyrrolidin-1-yl)propyl)-2,5-dihydro-1H-imidazole-2-carboxylate (A2-Iso5-2DC18); bis(2-(dodecyldisulfanyl)ethyl) 3,3′-((3-methyl-9-oxo-10-oxa-13,14-dithia-3,6-diazahexacosyl)azanediyl)dipropionate (BAME-O16B); 1,1′-((2-(4-(2-((2-(bis(2-hydroxydodecyl)amino)ethyl) (2-hydroxydodecyl)amino)ethyl) piperazin-1-yl)ethyl)azanediyl) bis(dodecan-2-ol) (C12-200); 3,6-bis(4-(bis(2-hydroxydodecyl)amino)butyl)piperazine-2,5-dione (cKK-E12); hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate (FTT5); (((3,6-dioxopiperazine-2,5-diyl)bis(butane-4, 1-diyl))bis(azanetriyl))tetrakis(ethane-2,1-diyl) (9Z,9′Z,9″Z,9′″Z,12Z,12′Z,12″Z,12′″Z)-tetrakis (octadeca-9,12-dienoate) (OF-Deg-Lin); TT3; N¹,N³,N⁵-tris(3-(didodecylamino)propyl)benzene-1,3,5-tricarboxamide; N1-[2-((1S)-1-[(3-aminopropyl)amino]-4-[di(3-aminopropyl)amino]butylcarboxamido)ethyl]-3,4-di[oleyloxy]-benzamide (MVL5); heptadecan-9-yl 8-((2-hydroxyethyl)(8-(nonyloxy)-8-oxooctyl)amino)octanoate (Lipid 5);

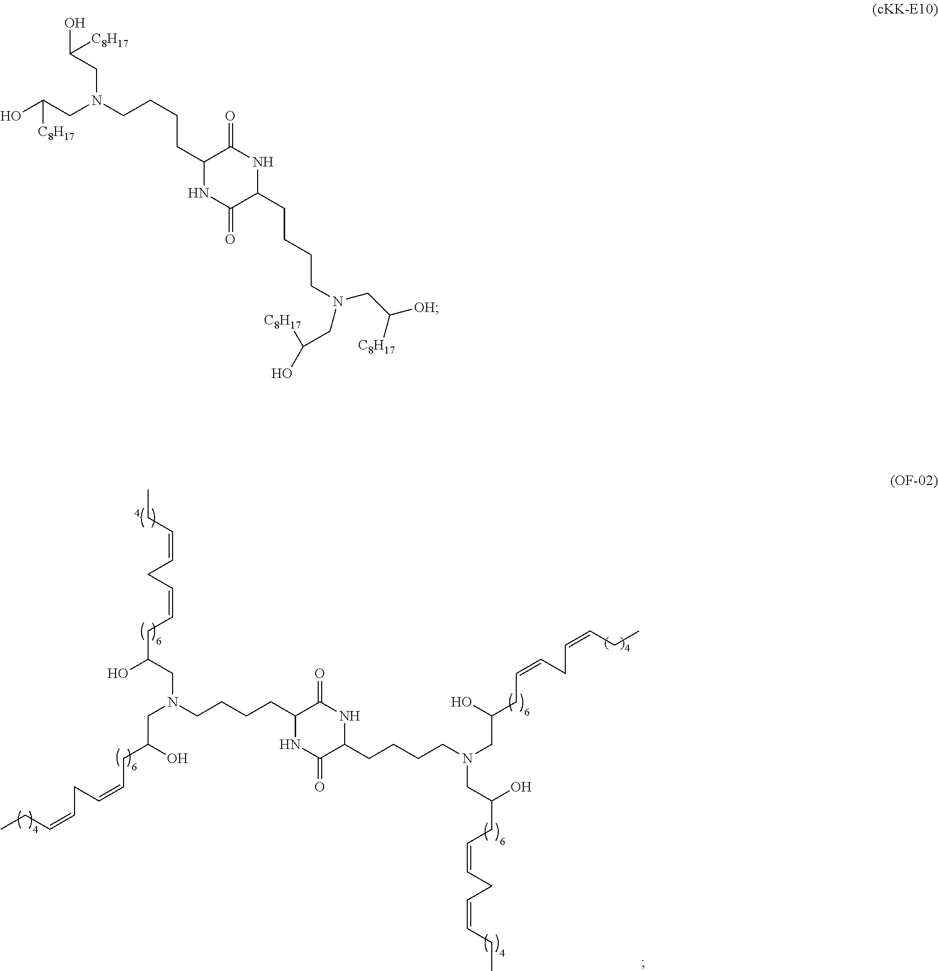

and combinations thereof; and/or
the neutral lipid is selected from the group comprising DSPC; DPPC; DMPC; POPC; DOPC; phosphatidylethanolamines, such as DOPE, DPPE, DMPE, DSPE, DLPE; sphingomyelins; ceramides, and combinations thereof, and/or
the sterol, or an ester thereof, is selected from the group consisting of cholesterol and its derivatives; ergosterol; desmosterol (3ß-hydroxy-5,24-cholestadiene); stigmasterol (stigmasta-5,22-dien-3-ol); lanosterol (8,24-lanostadien-3b-ol); 7-dehydrocholesterol (Δ5,7-cholesterol); dihydrolanosterol (24,25-dihydrolanosterol); zymosterol (5α-cholesta-8,24-dien-3ß-ol); lathosterol (5α-cholest-7-en-3ß-ol); diosgenin ((3β,25R)-spirost-5-en-3-ol); sitosterol (22,23-dihydrostigmasterol); sitostanol; campesterol (campest-5-en-3B-ol); campestanol (5a-campestan-3b-ol); 24-methylene cholesterol (5,24(28)-cholestadien-24-methylen-3ß-ol); cholesteryl margarate (cholest-5-en-3ß-yl heptadecanoate); cholesteryl oleate; cholesteryl stearate; and combinations thereof.

The LNPs may further comprise as lipid components at least one PEG-lipid.

The LNPs may comprise from about 0.5 to about 15%, or from about 0.5% to about 10%, or from about 0.8% to about 5%, or from about 1% to about 3%, or from about 1.5% to about 2% of said PEG-lipid, in % w/w relative to the total weight of the lipid components of said LNPs.

The PEG-lipid may be selected from the group consisting of PEG-DAG; DMG-PEG-2000; PEG-PE; PEG-S-DAG; PEG-S-DMG; PEG-cer; a PEG-dialkyoxypropylcarbamate; 2-[(polyethylene glycol)-2000]-N,N-ditetradecylacetamide (ALC-0159); and combinations thereof.

In some embodiments, the LNPs may comprise:
about 50% of ionizable cationic lipid, about 10% of neutral lipid, about 38.5% of cholesterol, and about 1.5% of PEG-lipid, or
about 46.3% of ionizable cationic lipid, about 9.4% of neutral lipid, about 42.7% of cholesterol, and about 1.6% of PEG-lipid, or
47.4% of ionizable cationic lipid, 10% of neutral lipid, 40.9% of cholesterol, and 1.7% of PEG-lipid, or
about 40% of ionizable cationic lipid, about 30% of neutral lipid, about 28.5% of cholesterol, and about 1.5% of PEG-lipid, or
about 50% of 9-heptadecanyl 8-{(2-hydroxyethyl)[6-oxo-6-(undecyloxy)hexyl]amino}octanoate (SM-102), about 10% of DSPC, about 38.5% of cholesterol, and about 1.5% of DMG-PEG-2000, or
about 46.3% of [(4-hydroxybutyl)azanediyl]di(hexane-6,1-diyl) bis(2-hexyldecanoate) (ALC-0315), about 9.4% of DSPC, about 42.7% of cholesterol, and about 1.6% of 2-[(polyethylene glycol)-2000]-N,N-ditetradecylacetamide (ALC-0159),
about 47.4% of [(4-hydroxybutyl)azanediyl]di(hexane-6,1-diyl) bis(2-hexyldecanoate) (ALC-0315), about 10% of DSPC, about 40.9% of cholesterol, and about 1.7% of 2-[(polyethylene glycol)-2000]-N,N-ditetradecylacetamide (ALC-0159), or
about 40% of cKK-E10, about 30% of DOPE, about 28.5% of cholesterol, and about 1.5% of DMG-PEG-2000, or
about 40% of ML7/OF-02, about 30% of DOPE, about 28.5% of cholesterol, and about 1.5% of DMG-PEG-2000,
in % w/w relative to the total weight of the lipid components of said LNPs.

In some embodiments, the nucleic acid contained in the LNPs may be an RNA. In some embodiments, the RNA may be an mRNA.

An mRNA may comprise a 5′Cap structure, a 5′UTR sequence, an ORF sequence, a 3′UTR sequence, and a poly(A) tail.

An mRNA may be at least 30 nucleotides in length.

In some embodiments, a nucleic acid may be or encode a therapeutic agent. The therapeutic agent may be a genome-editing polypeptide, a chemokine, a cytokine, a growth factor, an antibody, an enzyme, a structural protein, a blood protein, an hormone, a transcription factor, or an antigen.

In some embodiments, a nucleic acid may encode for an antigen. An antigen may be selected in the group comprising bacterial antigens, viral antigens, and tumour antigens. An antigen may be an antigen from a strain of Influenza A or Influenza B virus or from a Respiratory syncytial A or B virus, or from SARS-Cov2.

In some embodiments, the liquid composition comprising the LNPs further comprises at least one cryoprotectant. A cryoprotectant may be a polyol. A polyol may be selected from the group consisting of mannose, sucrose, lactose, trehalose, maltose, sorbitol, mannitol, glycerol, and inositol. A cryoprotectant may be trehalose.

In one of its objects, the present invention relates to frozen LNPs obtainable according to a method as disclosed herein.

In one of its objects, the present invention relates to freeze-dried LNPs obtainable according to a method as disclosed herein.

In one of its objects, the present invention relates to frozen LNPs comprising at least a nucleic acid and, at least, as lipid components, a cationic ionizable lipid, a neutral lipid, and a steroid alcohol, or an ester thereof, the frozen LNPs being in frozen micropellets. Such frozen LNPs may further comprise a PEG-lipid.

In one of its objects, the present invention relates to freeze-dried LNPs comprising at least a nucleic acid and, at least, as lipid components, a cationic ionizable lipid, a neutral lipid, and a steroid alcohol, or an ester thereof, said freeze-dried LNPs being in freeze-dried micropellets. Such freeze-dried LNPs may further comprise a PEG-lipid.

In one of its objects, the present invention relates to a method for manufacturing a medicament said method comprising at least the steps of preparing frozen or freeze-dried LNPs in accordance with a method as disclosed herein, the LNPs comprising at least a nucleic acid. The method for manufacturing a medicament may further comprise a step of resuspending the freeze-dried LNPs in a pharmaceutically acceptable solvent or thawing the frozen LNPs.

In one of its objects, the present invention relates to freeze-dried or frozen LNPs as disclosed herein and comprising at least nucleic acid, for use as a medicament.

In one of its objects, the present invention relates to frozen or freeze-dried LNPs as disclosed herein and comprising at least one nucleic acid encoding for an antigen from an Influenza A virus and/or an Influenza B virus for use in preventing or treating an Influenza A and/or an Influenza B virus infection.

In one of its objects, the present invention relates to frozen or freeze-dried LNPs as disclosed herein and comprising at least one nucleic acid encoding for an antigen from a Respiratory syncytial A virus and/or a Respiratory syncytial B virus for use in preventing or treating a Respiratory syncytial A virus and/or a Respiratory syncytial B virus infection.

In one of its objects, the present invention relates to frozen or freeze-dried LNPs as disclosed herein and comprising at least one nucleic acid encoding for an antigen from an Influenza A virus and/or an Influenza B virus for use as an immunogenic composition against an Influenza A virus and/or an Influenza B virus.

In one of its objects, the present invention relates to frozen or freeze-dried LNPs as disclosed herein and comprising at least one nucleic acid encoding for an antigen from a Respiratory syncytial A virus and/or a Respiratory syncytial B virus for use as an immunogenic composition against a Respiratory syncytial A virus and/or a Respiratory syncytial B virus.

In one of its objects, the present invention relates to a use of frozen or freeze-dried LNPs as disclosed herein and comprising at least a nucleic acid in the manufacture of a medicament.

In one of its objects, the present invention relates to a method for preventing and/or treating a disorder in an individual in need thereof, the method comprising at least the steps of:
resuspending freeze-dried LNPs as disclosed herein in a pharmaceutically acceptable solvent or thawing frozen LNPs, the frozen or freeze-dried LNPs comprising at least a nucleic acid presumed to be active against said disorder, to obtain thawed or resuspended LNPs and
administering to said individual the thawed or resuspended LNPs.

The invention will be more detailed in the following description.

### DETAILED DESCRIPTION

### Definitions

Unless otherwise defined herein, scientific and technical terms used in connection with the present invention shall have the meanings that are commonly understood by those of ordinary skill in the art. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, may provide one of skill with a general dictionary of many of the terms used in this disclosure. Exemplary methods and materials are described below, although methods and materials similar or equivalent to those described herein can also be used in the practice or testing of the present invention. In case of conflict, the present specification, including definitions, will control. Generally, nomenclature used in connection with, and techniques of, cell and tissue culture, molecular biology, virology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular.

Units, prefixes, and symbols are denoted in their Systéme International des Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure. Accordingly, the terms defined immediately below are more fully defined by reference to the specification in its entirety.

Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

It is understood that wherever aspects are described herein with the language “comprising,” otherwise analogous aspects described in terms of “consisting of” and/or “consisting essentially of” are also provided.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “approximately” or “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some embodiments, the term indicates deviation from the indicated numerical value by ±10%, 5%, 4%, 3%, 2%, 1%, 0.9%, 0.8%, 0.7%, 0.6%, 0.5%, 0.4%, 0.3%, ±0.2%, 0.1%, 0.05%, or ±0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%.

Depending on context, the term “polynucleotide” or “nucleotide” may encompass a singular nucleic acid as well as plural nucleic acids. Within the disclosure the term “nucleic acid”, “polynucleotide”, and “oligonucleotides” are used interchangeably. They refer to a polymeric form of at least two nucleotides, either deoxyribonucleotides or ribonucleotides, or analogs thereof. Nucleic acids may have any three-dimensional structure, and may perform any function, known or unknown. In some embodiments, a polynucleotide is an isolated nucleic acid molecule or construct, e.g., messenger RNA (mRNA) or plasmid DNA (pDNA). In some embodiments, a polynucleotide comprises a conventional phosphodiester bond. In some embodiments, a polynucleotide comprises a non-conventional bond (e.g., an amide bond, such as found in peptide nucleic acids (PNA)). The term “nucleic acid” may refer to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding a Factor VIII polypeptide contained in a vector is considered isolated for the purposes of the present disclosure. Further examples of an isolated polynucleotide include recombinant polynucleotides maintained in heterologous host cells or purified (partially or substantially) from other polynucleotides in a solution. Isolated RNA molecules include in vivo or in vitro RNA transcripts of polynucleotides of the present disclosure. Isolated polynucleotides or nucleic acids according to the present disclosure further include such molecules produced synthetically. In addition, a polynucleotide or a nucleic acid can include regulatory elements such as promoters, enhancers, ribosome binding sites, or transcription termination signals.

“Nucleic acid”, “polynucleotide”, and “oligonucleotides” may be linear or cyclic. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, closed-ended DNA (ceDNA), self-amplifying RNA (saRNA), stranded DNA (ssDNA), small interfering RNA (siRNA) and micro RNA (miRNA), recombinant polynucleotides, branched polynucleotides, plasmids, vectors, isolated DNA of any sequence, isolated RNA of any sequence, nucleic acid probes, and primers. A nucleic acid may comprise modified nucleotides, such as methylated nucleotides and nucleotide analogs. If present, modifications to the nucleotide structure may be imparted before or after assembly of the polymer. The sequence of nucleic acids may be interrupted by non-nucleotide components. A nucleic acid may be further modified after polymerization, such as by conjugation with a labeling component. The term “complement of a nucleic acid” denotes a nucleic acid molecule having a complementary base sequence and reverse orientation as compared to a reference sequence, such that it could hybridize with a reference sequence with complete fidelity. “Recombinant” as applied to a nucleic acid means that the nucleic acid is the product of various combinations of in vitro cloning, restriction and/or ligation steps, and other procedures that result in a construct that can potentially be expressed in a host cell.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced recombinant technology but is not necessarily translated from a designated nucleic acid sequence. It can be generated in any manner, including by chemical synthesis.

An “isolated” polypeptide or a fragment, variant, or derivative thereof refers to a polypeptide that is not in its natural milieu. No particular level of purification is required. For example, an isolated polypeptide can simply be removed from its native or natural environment. Recombinantly produced polypeptides and proteins expressed in host cells are considered isolated for the purpose of the disclosure, as are native or recombinant polypeptides which have been separated, fractionated, or partially or substantially purified by any suitable technique.

“Administer” or “administering,” as used herein refers to delivering to a subject a composition described herein, e.g., a chimeric protein. The composition, e.g., the chimeric protein, can be administered to a subject using methods known in the art. In particular, the composition can be administered intravenously, subcutaneously, intramuscularly, intradermally, or via any mucosal surface, e.g., orally, sublingually, buccally, nasally, rectally, vaginally or via pulmonary route. In some embodiments, the administration is intravenous. In some embodiments, the administration is subcutaneous. In some embodiments, the administration is self-administration. In some embodiments, a parent administers the chimeric protein to a child. In some embodiments, the chimeric protein is administered to a subject by a healthcare practitioner such as a medical doctor, a medic, or a nurse.

The term “antigen” comprises any molecule, for example a peptide or protein, which comprises at least one epitope that will elicit an immune response and/or against which an immune response is directed. For example, an antigen is a molecule which, optionally after processing, induces an immune response, which is for example specific for the antigen or cells expressing the antigen. After processing, an antigen may be presented by MHC molecules and reacts specifically with T lymphocytes (T cells). Thus, an antigen or fragments thereof should be recognizable by a T cell receptor and should be able to induce in the presence of appropriate co-stimulatory signals, clonal expansion of the T cell carrying the T cell receptor specifically recognizing the antigen or fragment, which results in an immune response against the antigen or cells expressing the antigen.

According to the present disclosure, any suitable antigen may be envisioned which is a candidate for an immune response. An antigen may correspond to or may be derived from a naturally occurring antigen. Such naturally occurring antigens may include or may be derived from allergens, viruses, bacteria, fungi, parasites and other infectious agents and pathogens or an antigen may also be a tumor antigen.

The expression “ionizable cationic lipid” refers to lipids containing one or more groups which can be protonated at physiological pH but may deprotonated at a pH above 8, 9, 10, 11, or 12. The ionizable cationic group may contain one or more protonatable amines which are able to form a cationic group at physiological pH. The cationic ionizable lipid compound may also further comprise one or more lipid components such as two or more fatty acids with C₆-C₂₄ alkyl or alkenyl carbon groups. These compounds may be a dendrimer, a dendron, a polymer, or a combination thereof.

The expression “lipid component” refers to a group of organic compounds that include, but are not limited to, esters of fatty acids and are generally characterized by being poorly soluble in water, but soluble in many organic solvents. Lipid is a generic term encompassing fats, fatty oils, essential oils, waxes, phospholipids, glycolipids, sulfolipids, aminolipids, chromolipids (lipochromes), and fatty acids. Within the disclosure, “lipid” encompasses neutral lipids, steroid alcohol or ester thereof, and PEGylated lipids.

The expression “lipid nanoparticles” (LNPs) refers to particles having at least one dimension on the order of nanometers (e.g., 10-800 nm, and for example from about 80 to about 200 nm as measured by Nanoparticle Tracking Analysis (NTA)) which may be formulated with at least one of the lipid components as disclosed herein. In some embodiments, LNPs are included in a formulation that can be used to deliver an active agent or therapeutic agent, such as a nucleic acid to a target site of interest (e.g., cell, tissue, organ, tumor, and the like). Such lipid nanoparticles typically comprise a lipid component as disclosed herein.

The expression “frozen lipid nanoparticles” refers to a liquid composition of LNPs which has been subjected to temperature conditions under which its solvent component has been solidified.

The expression “freeze-dried lipid nanoparticles” refers to a liquid composition of LNPs which has been frozen and then subjected to drying conditions under which its solvent component has been evaporated.

The terms “micropellet(s)” or “microbeads” are used interchangeably are intended to refer to particles with a tendency to be generally spherical/round in the micrometer range. Frozen or freeze-dried micropellets may have with a mean value for the diameters chosen from a range of about 200 to about 1500 micrometers (μm), with a selectable, preferably narrow particle size distribution of about ±50 μm around the chosen value.

The expression “cationic ionizable lipid” refers to lipids containing one or more groups which can protonated at physiological pH but may deprotonated at a pH above 8, 9, 10, 11, or 12. The ionizable cationic group may contain one or more protonatable amines which are able to form a cationic group at physiological pH. The cationic ionizable lipid compound may also further comprise one or more lipid components such as two or more fatty acids with C₆-C₂₄ alkyl or alkenyl carbon groups. These compounds may be a dendrimer, a dendron, a polymer, or a combination thereof

The expression “neutral lipid” refers to any lipid components that is either not ionizable or is a neutral zwitterionic compound at a selected pH, for example at physiological pH. Such lipids include, but are not limited to, phosphatidylcholines, phosphatidylethanolamines sphingomyelins (SM), or neutral sphingolipids such as ceramides. Neutral lipids may be synthetic or naturally derived.

The expressions “PEG-lipid” or “PEGylated lipid” are used interchangeably and intend to refer to a molecule comprising both a lipid portion and a polyethylene glycol portion. PEG-lipid are known in the art and include 1-(monomethoxy-polyethyleneglycol)-2,3-dimyristoylglycerol (PEG-DMG) and the like.

Within the disclosure, the term “prilling” intends to refer to a process for solidifying droplets of a liquid material falling in a cooling material or against an upward stream of cooling material such as a cooling gas or a refrigerant.

Within the disclosure, the term “significantly” used with respect to change intends to mean that the observe change is noticeable and/or it has a statistic meaning.

The expression “spray-freeze drying” intends to refer a process in which a feed solution is broken down into droplets, the droplets are then frozen by contact with a low-temperature medium, and then the frozen droplets are transferred into a freeze-dryer to sublimate water and to obtain a dried powder. The dried powder may be dried micropellets.

Within the disclosure, the term “substantially” used in conjunction with a feature of the disclosure intends to define a set of embodiments related to this feature which are largely but not wholly similar to this feature.

The expressions “steroid alcohol” or “sterol” are used interchangeably and intend to refer to a group of lipids comprised of a sterane core bearing a hydroxyl moiety. As example of steroid alcohol, one may cite cholesterol, campesterol, sitosterol, stigmasterol and ergosterol. Esters of steroid alcohol or of sterol refer to ester of carboxylic acid with the hydroxyl group of the steroid alcohol. Suitable carboxylic acid comprises, further to the carboxyl moiety, a saturated or unsaturated, linear or branched, alkyl group. In some embodiments the alkyl group may be a C₁-C₂₀ alkyl group. In other embodiments, the carboxylic acid may be a fatty acid.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention. All publications mentioned herein are incorporated herein by reference to disclose and describe the methods and/or materials in connection with which the publications are cited.

The list of sources, ingredients, and components as described hereinafter are listed such that combinations and mixtures thereof are also contemplated and within the scope herein.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All lists of items, such as, for example, lists of ingredients, are intended to and should be interpreted as Markush groups. Thus, all lists can be read and interpreted as items “selected from the group consisting of” the list of items “and combinations and mixtures thereof.”

Referenced herein may be trade names for components including various ingredients utilized in the present disclosure. The inventors herein do not intend to be limited by materials under any particular trade name. Equivalent materials (e.g., those obtained from a different source under a different name or reference number) to those referenced by trade name may be substituted and utilized in the descriptions herein.

### Spraying, Freezing and Drying

The present invention relates to methods for spray-freezing or spray-freeze-drying lipid nanoparticles (LNPs). The LNPs may comprise at least a nucleic acid and, at least, as lipid components, a cationic ionizable lipid, a neutral lipid, and a steroid alcohol, or an ester thereof, wherein said method comprises the steps of:

The spray-freezing methods as disclosed herein may comprise the steps of:
a) providing a liquid composition comprising said LNPs,
b) spraying the composition of step a) in conditions suitable for obtaining liquid droplets, and
c) freezing the liquid droplets obtained at step b) to obtain frozen LNPs.

The frozen LNPs may be obtained in frozen micropellets.

The spray-freeze drying methods as disclosed herein may comprise the steps of:
a) providing a liquid composition comprising said LNPs,
b) spraying the composition of step a) in conditions suitable for obtaining liquid droplets,
c) freezing the liquid droplets obtained at step b) to obtain frozen LNPs, and
d) drying the frozen LNPs obtained at step c) under conditions suitable to obtain freeze-dried LNPs.

The freeze-dried LNPs may be obtained in freeze-dried micropellets. Micropellets may be obtained by prilling and drying.

Freeze-drying, also known as lyophilization, is a process commonly used for drying labile products such as, for example, pharmaceuticals, biological materials, e.g., proteins, enzymes, microorganisms, and in general any thermo- and/or hydrolysis-sensitive material.

### Spraying

The spraying may be carried out with an electromagnetic droplet stream generator, a piezoelectric droplets stream generator, a hydraulic droplets aerosol generator, a pneumatic nozzle, a ultrasonic spray nozzle, a thermal droplets stream generator, or an electrohydrodynamic droplets (EHD) generator.

In some embodiments, the spraying may be carried out with piezoelectric droplet stream generator.

### Electromagnetic or Piezoelectric Droplet Stream Generation

Prilling, also known as a laminar jet break-up technique, allows generation and solidification of calibrated monodisperse droplets of liquid. Prilling may be carried out by electromagnetic or piezoelectric droplet stream generation and freezing of the droplets.

The main principle of electromagnetic or piezoelectric droplet-stream generators is based on Rayleigh disintegration of a liquid jet exiting from the orifice of a capillary with the use of a mechanical vibration obtained by a electromagnet or a piezoceramic oscillator. Lord Rayleigh proposed a model for Newtonian fluids (Rayleigh L, Proc. London Math. Soc. 1978. 10, 4-13). For aqueous solutions exiting from small circular orifices at low pressure, the formation of small droplets with diameters down to a few micrometers is limited by the surface tension and the adhesion of the liquid to the nozzle wall. By piezoelectric excitation, the breakup length of the liquid jet can be shortened and the signal type (e.g., sinusoidal, rectangular), frequency and amplitude affect both the mean size and the uniformity of the droplets.

The optimal wavelength for the fastest growing disturbance and jet break-up is given by:

$${λ}_{o p t} = π \sqrt{2} {d}_{j} \sqrt{1 + \frac{3 η}{\sqrt{ρ σ {d}_{j}}}}$$

Where λₒₚₜ is the optimal wavelength for jet break-up, dⱼ, is the diameter of the jet, η is the viscosity of the fluid, ρ is the density of the fluid and σ is the surface tension of the fluid.

The diameter d of the droplets formed can be calculated by:

$$d = \sqrt[3]{1.5 * {d}_{j}^{2} * {λ}_{o p t}}$$

The frequency f to apply to the fluid to achieve the desired results is related to the jet velocity (and therefore the flow rate of the fluid) u; and the wavelength by:

$$λ = \frac{{u}_{j}}{f}$$

Therefore, optimal conditions can be calculated knowing process parameters and fluid characteristics. A range of frequencies and jet velocities exist to form uniform droplets depending on the nozzle diameter, rheology of the fluid and surface tension (Meesters G., 1992. Mechanisms of droplet formation. Delft University Press, Delft, NL).

Suitable working frequencies can also be determined experimentally by visual assessment of the stability of the droplet formation. Standard prilling equipment are equipped with light stroboscope to observe the droplet formation: for a given product and given working conditions, one can adjust manually the frequency until observing a stable and still droplets chain with this stroboscope light.

Prilling allows generating monodisperse calibrated droplets with diameter ranging for example from about 200 μm to about 1500 μm, or from about 300 μm to about 600 μm with a narrow size distribution of +/−25%, or +/−10%.

The electromagnetic or the piezoelectric droplet stream generator is a nozzle. Suitable nozzle and multinozzle systems have been developed for aseptic prilling applications, such as disclosed in Brandenberger et al., J. Biotechnol., 1998, 63, 73-80, or in WO 2016/012414 A1.

The nozzle may have an outlet aperture of a diameter from about 250 μm to about 400 μm and may be of about 300 μm.

The prilling process may be adapted to viscous liquids. Acceptable viscosity may be approximately 300 mPa·s.

Temperatures in the feeding reservoir containing the liquid composition comprising the LNPs and in the nozzle have to be controlled in order to avoid components or solvent crystallization before droplets formation. The person skilled in the art of formulation knows how to adjust different components concentrations in the stabilizing formulation in order to avoid non-controlled crystallization and viscosities above the given limit, taking into account eventual interactions between excipients.

Examples of nozzles for piezoelectric droplet stream generator are disclosed in Wanning at al., (Int J Pharm. 2015; 488(1-2):136-153), the content of which is incorporated by reference. Examples of nozzles for electromagnetic droplet stream generator are disclosed in in WO 2016/012414 A1), the content of which is incorporated by reference.

### Other Spraying Methods

Other spraying methods may be suitable for the methods as disclosed herein, such as spraying with hydraulic droplets aerosol generator, pneumatic nozzle, ultrasonic spray nozzle, thermal droplets stream generator, or electrohydrodynamic droplets (EHD) generator. Such methods are disclosed in Adali et al., Processes. 2020; 8(6) and in Wanning et al., Int J Pharm. 2015; 488(1-2):136-153), the content of which is incorporated by reference.

With hydraulic nozzles, sprays are generated by forcing the fluid through an orifice. The required energy is provided by converting the pressure into kinetic energy, and the droplet size varies as a function of the feed rate and viscosity, and the spraying pressure.

With pneumatic nozzles, the atomization energy is provided by a compressed gas flow (usually air) that interacts with the liquid and produces a shear field that results in a wide range of droplet sizes. These devices are also known as multi-fluid nozzles. For example, in a two-fluid nozzles, the liquid feed and the compressed gas are fed into the nozzle to produce a shear field.

With ultrasonic nozzles, the liquid is broken into fine droplets when a high-frequency electrical signal is converted into mechanical energy and transferred into the liquid. Typically, ultrasonic nozzles consist of two piezoelectric transducers that receive electrical input placed between two electrodes. This causes simultaneous mechanical expansion and contraction of the transducers, resulting in ultrasonic vibrations sent to the nozzle tip in order to atomize the feed. The droplet size depends on the operating frequency and feed flow rate. The use of such a device allows advanced control over the particle size and provides a narrow droplet size distribution.

### Freezing

Various techniques known in the art may be used to freeze the droplets. Freezing is defined as the solidification of solvent and the rendering of most or all of the solute phase into a rigidified state by the removal of heat.

The freezing of the liquid droplets may be obtained by contacting the droplets with a freezing gas, a freezing liquid, or a freezing surface. The step of freezing may be carried out by spraying the liquid droplets into a cryogenic atmosphere, with compressed carbon dioxide, into vapor over a cryogenic liquid, into a cryogenic liquid, or onto a cold solid surface.

In some embodiments, the step of freezing may be carried out by spraying the liquid droplets into a cryogenic atmosphere.

### Freezing in a Cryogenic Atmosphere

In the methods as disclosed herein, the step of freezing may be carried out by spraying the liquid droplets into a cryogenic atmosphere.

In atmospheric freezing, the heat sink is gaseous, at ambient pressure with a nearly uniform temperature sufficiently low to induce the formation of ice nuclei in the solution. The frictional stress is generally low and the size and the approximately spherical shape of the droplets are not altered as they solidify. Under these conditions, the cooling rate is limited by the rate of energy transfer across the droplet surface, which depends upon the slip velocity.

In some embodiments, the freezing may be achieved by letting the droplets free-falling in a cryogenic chamber in which the temperature is maintained in range from about −100° C. to about −160° C., for example at about −110° C. or −105° C. by a freezing medium. A freezing medium may be introduced in the freezing chamber by direct injection/nebulization of a freezing gas all along the liquid droplets pathway. Alternatively, the freezing medium may be introduced as a flowing current of a freezing gas in counter current to the flow of the liquid droplets, or by maintaining a static freezing gas in the chamber at a pressure above the atmospheric pressure (for example the overpressure may be from 1.1 to 1.5 the atmospheric pressure). In some embodiments, the freezing medium is introduced in the freezing chamber by direct injection/nebulization of a freezing gas all along the liquid droplets pathway.

In some embodiments, a spatial temperature profile is configured in the cryogenic chamber. For example, a spatial temperature profile in the chamber may be configured and maintained such as a temperature ranging from −40° C. to −60° C., for example from −50° C. and −60° C., is maintained in a top area and a temperature ranging from −150° C. to −192° C., for example from −150° C. and −160° C., is maintained in a bottom area of the tower.

The temperatures in the cryogenic chamber may optionally be maintained or varied/cycled throughout between about −50° C. to −190° C.

The liquid droplets freeze during their free-fall in the cryogenic chamber to form calibrated frozen particles. The minimum falling height to freeze the droplets (i.e., ice crystals formation that solidifies the pellets) into frozen droplets may depend on the size of the liquid droplets to be frozen, the method used to freeze the droplets, i.e., direct injection/nebulization of freezing gas in the chamber, or counter current of freezing gas, or static freezing gas at a pressure above the atmospheric pressure).

The frozen droplets may have a diameter ranging from about 200 μm to about 1500 μm, or from about 200 μm to about 800 μm, or from about 300 μm to about 600 μm, or at about 500 μm. Size of the particles may be measured with a particle size analyzer or an imaging particle size analyzer.

Frozen droplets obtained by such method may be referred to frozen micropellets.

For forming freezing droplets into round micropellets with sizes/diameters in the range of 100-800 μm, an appropriate height of the cryogenic chamber may be between 1-2 m (meters), while forming freezing droplets into pellets with a size range up to 1500 μm (micrometers) the cryogenic chamber may be between about 2-3 m wherein the diameter of the cryogenic chamber can be between about 50-150 cm for a height of 200-300 cm.

A freezing medium may have a temperature below −110° C.

A freezing medium may be liquid or vapor nitrogen, liquid or vapor CO₂, or liquid air and/or vapor of thereof.

### Other Freezing Methods

Other freezing methods may be suitable for the methods as disclosed herein, such as by spraying the liquid droplets with compressed carbon dioxide, into vapor over a cryogenic liquid, into a cryogenic liquid, or onto a cold solid surface.

Such methods are disclosed in Adali et al., Processes. 2020; 8(6) and in Wanning et al., Int J Pharm. 2015; 488(1-2):136-153), the content of which is incorporated by reference.

In spray-freezing with compressed carbon dioxide, the temperature of aqueous sprays can also be reduced below the freezing point by Joule-Thompson cooling of co-expanding carbon dioxide.

Freezing by spraying into vapor over a cryogenic liquid (SFV) may be carried out by spraying droplets into a gaseous freezing medium above the freezing point of the liquid freezing medium and sediment through the vapor layer onto the surface of the liquid freezing medium. Supercooling and freezing may occur in the supernatant gas and vapor or upon contact with the condensed refrigerant. Since the velocity of small droplets decreases rapidly due to atmospheric braking, frictional stresses remain low and the freezing conditions are similar to those upon atmospheric freezing.

Spray-freezing into liquid (SFL) may allow achieving high freezing rates since the solution to be frozen is directly injected at high flow rates into a cryogenic liquid. Under these conditions, frictional stresses are high and the fluid dynamic conditions are not well defined. The particles formed are frequently small fragments. Alternatively, the solution may be dripped or sprayed at a lower rate from a nozzle into the liquid freezing medium. If the density of the solution to be frozen is lower than that of the cryogenic fluid, it can also be injected from the bottom of the freezing vessel and the frozen particles are skimmed off the surface.

High cooling rates and uniform particulate materials can be also produced by spraying or dripping liquids on a cold solid surface. Thus, the freezing rate is accelerated compared to volatile cryogenic liquids because the Leidenfrost effect is avoided, in which a vapor layer limits the transfer of thermal energy to the heat sink.

Also in those methods, a freezing medium may have a temperature below −110° C. A freezing medium may be liquid nitrogen, liquid CO₂ or liquid air and/or vapor of thereof.

After the freezing step, the frozen droplets may be then collected and transferred in a freeze-drier. Alternatively, they may be stored until they are freeze-dried. Such storage may be carried on pre-cooled trays in conditions allowing keeping them below the glass transition Tg′ of their cryo-concentrated phase to avoid any melting or aggregation of the frozen droplets. For example, for a Tg′ value ranging from −10° C. to −45° C., the storage temperature should be at least equal or less than −50° C. Frozen LNPs disclosed herein may be stored a −70° C.

Frozen droplets are stored in conditions suitable for avoiding any melting or aggregation of the frozen droplets.

### Drying (or Freeze-Drying)

The drying (or freeze-drying) may be carried out by rotary drum vacuum lyophilization, atmospheric drying with a flow of cold air, vacuum chamber lyophilization, or vacuum tunnel lyophilization.

“Vacuum” is understood as denoting a low pressure or an under-pressure, that is below an atmospheric pressure, as is known to the skilled person. Vacuum conditions as used herein may mean a pressure as low as 10 millibar, or 1 millibar, or 500 microbar, or 1 microbar. It should be noted that lyophilization may generally be performed in different pressure regimes and may, for example, be performed under atmospheric pressure.

In some embodiments, the drying may be carried out by rotary drum vacuum lyophilization. In some embodiments, the drying may be carried out by lyophilization in a vacuum chamber.

The obtained frozen micropellets may be dried by being subjected to sublimations conditions. Sublimations conditions allows evaporating the frozen solvent, i.e., from frozen state to gaseous state, at low heating temperature and under vacuo.

### Drying by Rotary Drum Vacuum Lyophilization

In some embodiments, the drying step may be carried in a vacuum rotary drum drier. A suitable vacuum rotary drier is disclosed in WO 2013/050157 A1, WO 2013/050158 A1, WO 2013/050159 A1, in Adali et al., Processes. 2020; 8(6), or in Wanning et al., Int J Pharm. 2015; 488(1-2):136-153), the content of which is incorporated by reference.

A suitable rotary drier may be placed in a vacuum chamber.

The drum of the rotary drier may comprise a temperature-controllable inner wall surface, for example by means of a double-wall. Additionally, or alternatively, other means for heating the micropellets during a lyophilization process may be provided, such as, for example, microwave or infra-red heating.

The inner wall surface temperatures of a drier may be controllable within a range of about −60° C. to +125° C.

During freeze-drying, the drum of the rotary drier may be rotated in order to maximize the inner wall surface available for the evaporation of solvent. Typical rotation velocities during a freeze-drying process may include, but are not limited to, between about 0.5-10 rotations per minute (rpm), for example between 1-8 rpm.

The freeze-dried droplets may have a diameter ranging from about 200 μm to about 1500 μm, or from about 200 μm to about 800 μm, or from about 300 μm to about 600 μm, or at about 500 μm.

Freeze-dried droplets obtained by such method may be referred to freeze-dried micropellets.

### Other Drying Methods

Other drying methods may be suitable for the methods as disclosed herein, such as atmospheric drying with a flow of cold air, vacuum chamber lyophilization, or vacuum tunnel lyophilization. Those methods are suitable alternative to drying in rotary drum.

Such methods are disclosed in Adali et al., Processes. 2020; 8(6) and in Wanning et al., Int J Pharm. 2015; 488(1-2):136-153), the content of which is incorporated by reference.

In atmospheric freeze drying cold and dry air or gas at atmospheric pressure passes over the frozen droplets and removes solvent from their surface. With particulate drying materials, the process gas can either rise through a bed of frozen droplets or, if the frozen droplets rest on a permeable support, pass through it in a descending flow. At sufficiently fast upstream flow rates, fluidized or spouting beds are formed, depending upon the inertia of the frozen droplets, the geometry of the chamber and the gas dynamics. In downstream drying, the gas percolates mainly through the voids between the frozen droplets.

In vacuum chamber lyophilization and vacuum tunnel lyophilization, the frozen droplets are placed in a low-atmospheric pressure (vacuum) environment and low temperature. Application of vacuum during the drying process allows removing the solvent. Primary drying removes the water from formulations by sublimation of ice, and then secondary drying removes the unfrozen bound water.

In vacuum chamber lyophilization, the frozen droplets are placed on trays and dried in layers, the sublimation rate is determined by a bimodal pore size distribution, where the short-range diffusion of free solvent molecules is determined by the internal pores and their connectivity. The sublimation energy is provided by conduction from the lower heating plate and/or by radiation from a radiating shelf. In some embodiments, the drying is carried in vacuum chamber lyophilization.

Vacuum tunnel lyophilization allows reducing the drying time and increasing the energy efficiency of lyophilization is by reducing the thickness of the layer of frozen droplets and supplying the sublimation energy by infrared or microwave radiation. The frozen droplets are placed on trays, which are passed through entry locks into a vacuum tunnel and unloaded through exit locks in a quasi-continuous process.

For example, once a freeze-drier (vacuum chamber lyophilization and vacuum tunnel lyophilization) is loaded with the trays, vacuum is pulled in the chamber or the tunnel to initiate conventional freeze-drying (sublimation of the ice) of the frozen droplets.

The following freeze-drying parameters are an example of what may be used for a formulation which a Tg′ ranging from about −30° C. to about −45° C.:

Primary drying: shelf temperature equal to −35° C., pressure equal to 50 μbars during 10 h.

Secondary drying: shelf temperature equal to 20° C., pressure equal to 50 μbars during 3 h.

Freeze-drying cycle has to be designed in order to get residual moistures preferentially lower than 3%. However, the moisture content can be optimized at higher value, on a case-by-case basis, if the stability of the material to be freeze-dried requires it.

The freeze-dried droplets, or micropellets, may be then collected in bulk. Storage conditions are suitable for dry, friable and hygroscopic particles. Bulk of freeze-dried droplets may be then filled into vials using dry powder filling technologies known in the art.

### Lipid Nanoparticles, and Manufacturing Processes

Suitable lipid components for LNPs as disclosed herein may comprise, as lipid component, at least: one ionizable lipid, one neutral lipid, and one steroid alcohol or ester thereof.

Optionally, at least one PEG-lipid may also be implemented.

### Ionizable Cationic Lipids

LNPs as disclosed herein may comprise at least one ionizable cationic lipid.

A ionizable cationic lipid may containing one or more groups which can be protonated at physiological pH but may deprotonated at a pH above 8, 9, 10, 11, or 12. The ionizable cationic group may contain one or more protonatable amines which are able to form a cationic group at physiological pH. The cationic ionizable lipid may also further comprise one or more lipid components such as two or more fatty acids with C₆-C₂₄ alkyl or alkenyl carbon groups. These compounds may be a dendrimer, a dendron, a polymer, or a combination thereof

In some embodiments, ionizable cationic lipid may comprises at least one protonatable amine moiety.

A suitable ionizable cationic lipid may be a ionizable cationic lipid from U.S. Pat. No. 9,512,073 or in U.S. Pat. No. 10,201,618, the content of which is herein incorporated by reference.

A suitable ionizable cationic lipid may be selected from the group comprising [(6Z,9Z,28Z,31Z)-heptatriaconta-6,9,28,31-tetraen-19-yl] 4-(dimethylamino)butanoate (D-Lin-MC3-DMA); 2,2-dilinoleyl-4-dimethylaminoethyl-[1,3]-dioxolane (Dlin-KC2-DMA); 1,2-dilinoleyloxy-N,N-dimethyl-3-aminopropane (Dlin-DMA); di((Z)-non-2-en-1-yl) 9-((4-(dimethylamino)butanoyl)oxy)heptadecanedioate (L319); 9-heptadecanyl 8-{(2-hydroxyethyl)[6-oxo-6-(undecyloxy)hexyl]amino}octanoate (SM-102); [(4-hydroxybutyl)azanediyl]di(hexane-6,1-diyl) bis(2-hexyldecanoate) (ALC-0315); [3-(dimethylamino)-2-[(Z)-octadec-9-enoyl]oxypropyl] (Z)-octadec-9-enoate (DODAP); 2,5-bis(3-aminopropylamino)-N-[2-[di(heptadecyl)amino]-2-oxoethyl]pentanamide (DOGS); [(3S,8S,9S,1OR,13R,14S,17R)-10,13-dimethyl-17-[(2R)-6-methylheptan-2-yl]-2,3,4,7,8,9,11,12,14,15,16,17-dodecahydro-1H-cyclopenta[a]30henanthrene-3-yl] N-[2-(dimethylamino)ethyl]carbamate (DC-Chol); tetrakis(8-methylnonyl) 3,3′,3″,3′″-(((methylazanediyl) bis(propane-3,1 diyl))bis (azanetriyl))tetrapropionate (306Oi10); decyl (2-(dioctylammonio)ethyl) phosphate (9A1P9); ethyl 5,5-di((Z)-heptadec-8-en-1-yl)-1-(3-(30henanthren-1-yl)propyl)-2,5-dihydro-1H-imidazole-2-carboxylate (A2-Iso5-2DC18); bis(2-(dodecyldisulfanyl)ethyl) 3,3′-((3-methyl-9-oxo-10-oxa-13,14-dithia-3,6-diazahexacosyl)azanediyl)dipropionate (BAME-016B); 1,1′-((2-(4-(2-((2-(bis(2-hydroxydodecyl)amino)ethyl) (2-hydroxydodecyl)amino)ethyl) piperazin-1-yl)ethyl)azanediyl) bis(dodecan-2-ol) (C12-200); 3,6-bis(4-(bis(2-hydroxydodecyl)amino)butyl)piperazine-2,5-dione (cKK-E12); hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate (FTT5); (((3,6-dioxopiperazine-2,5-diyl)bis(butane-4, 1-diyl))bis(azanetriyl))tetrakis(ethane-2,1-diyl) (9Z,9′Z,9″Z,9′″Z,12Z,12′Z,12″Z,12′″Z)-tetrakis (octadeca-9,12-dienoate) (OF-Deg-Lin); TT3; N¹,N³,N⁵-tris(3-(didodecylamino)propyl)benzene-1,3,5-tricarboxamide; N1-[2-((1S)-1-[(3-aminopropyl)amino]-4-[di(3-aminopropyl)amino]butylcarboxamido)ethyl]-3,4-di[oleyloxy]-benzamide (MVL5); 30henanthren-9-yl 8-((2-hydroxyethyl)(8-(nonyloxy)-8-oxooctyl)amino)octanoate (Lipid 5);

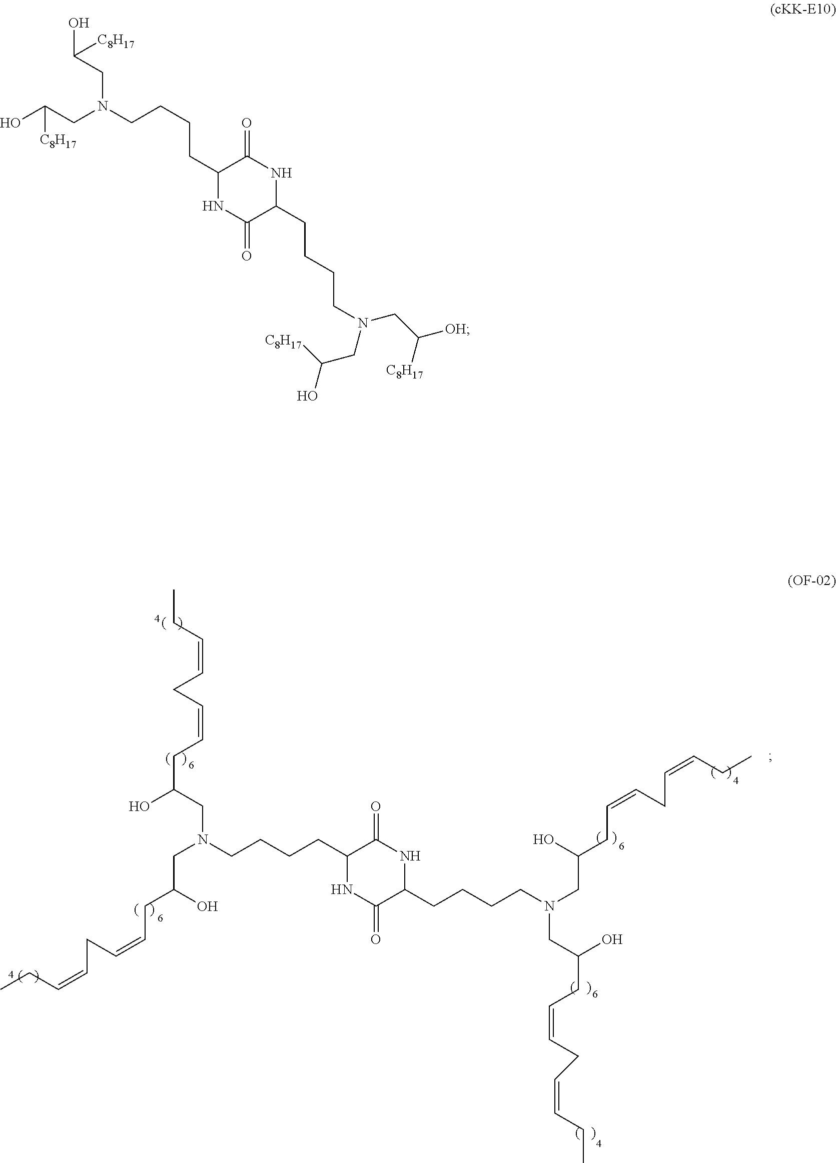

and combinations thereof.

The ionizable cationic lipid OF-02 is disclosed in particular in the PCT application WO 2022/099003, the content of which being incorporated by reference.

The LNPs may comprise from about 20 to about 60%, or from about 25% to about 60%, or from about 30% to about 55%, or from about 40% to about 55%, or from about 40% to about 50%, of ionizable cationic lipid, in % w/w relative to the total weight of the lipid components of said LNPs.

In one embodiment, a suitable ionizable cationic lipid may be (6Z,9Z,28Z,31Z)-heptatriacont-6,9,28,31-tetraene-19-yl 4-(dimethylamino)butanoate or Dlin-MC3-DMA (also known as MC3).

In one embodiment, a suitable ionizable cationic lipid may be 9-heptadecanyl 8-{(2-hydroxyethyl)[6-oxo-6-(undecyloxy)hexyl]amino}octanoate (SM-102), for example present in amount of about 50% in % w/w relative to the total weight of the lipid components of said LNPs.

In one embodiment, a suitable ionizable cationic lipid may be [(4-hydroxybutyl)azanediyl]di(hexane-6,1-diyl) bis(2-hexyldecanoate) (ALC-0315), for example present in amount of about 46.3% or about 47.4% in % w/w relative to the total weight of the lipid components of said LNPs.

In one embodiment, a suitable ionizable cationic lipid may be cKK-E10, for example present in amount of about 40% in % w/w relative to the total weight of the lipid components of said LNPs.

In one embodiment, a suitable ionizable cationic lipid may be OF-02, for example present in amount of about 40% in % w/w relative to the total weight of the lipid components of said LNPs.

### Neutral Lipids

LNPs as disclosed herein may comprise at least one neutral lipid. The presence of neutral lipids may improve structural stability of the lipid nanoparticles. The neutral lipid can be appropriately selected in view of the delivery efficiency of nucleic acid.

The neutral lipids are distinct from the ionizable cationic lipid as disclosed herein. Neutral lipids are either not ionizable or are neutral zwitterionic compounds at a selected pH.

Suitable neutral lipids useful for the LNPs may be selected from the group consisting of phosphatidylcholines, phosphatidylethanolamines, sphingomyelins, and ceramides.

Phosphatidylcholines and phosphatidylethanolamines are zwitterionic lipids. Sphingomyelins and ceramides are not ionizable lipids.

A phosphatidylcholine may be DSPC (1,2-distearoyl-sn-glycero-3-phosphocholine), DPPC (1,2-dipalmitoyl-sn-glycero-3-phosphocholine), DMPC (1,2-dimyristoyl-sn-glycero-3-phosphocholine), POPC (1-palmitoyl-2-oleoyl-sn-glycero-3-phosphocholine), DOPC (1,2-dioleoyl-sn-glycero-3-phosphocholine).

A phosphatidylethanolamine may be DOPE (1,2-dioleyl-sn-glycero-3-phosphoethanolamine), DPPE (1,2-dipalmitoyl-sn-glycero-3-phosphoethanolamine), DMPE (1,2-dimyristoyl-sn-glycero-3-phosphoethanolamine), DSPE (1,2-distearoyl-s/i-glycero-3-phosphoethanolamine), DLPE (1,2-dilauroyl-SM-glycero-3-phosphoethanolamine), 16-O-monomethyl PE, 16-O-dimethyl PE, 18-1-trans PE, or I-stearoyl-2-oleoyl-phosphatidyethanolamine (SOPE).

A neutral lipid may be selected from the group consisting of phosphatidylcholines, such as DSPC, DPPC, DMPC, POPC, DOPC; phosphatidylethanolamines, such as DOPE, DPPE, DMPE, DSPE, DLPE; sphingomyelins; ceramides, and combinations thereof.

In one embodiment, a neutral lipid may be DSPC, DOPC, and DOPE, and for example may be DSPC or DOPE.

In one embodiment, a neutral lipid may be DSPC.

The LNPs may comprise from about 5 to about 50%, or from about 5% to about 45%, from about 9% to about 40%, from about 9% to about 30% of neutral lipid, in % w/w relative to the total weight of the lipid components of said LNPs.

In one embodiment, a suitable neutral lipid may be DSP, for example present in amount of about 10% in % w/w relative to the total weight of the lipid components of said LNPs.

In one embodiment, a suitable neutral lipid may be DOPE, for example present in amount of about 30% in % w/w relative to the total weight of the lipid components of said LNPs.

Neutral lipids may be present in LNPs as disclosed herein in a molar ratio ionizable cationic lipid:neutral lipid ranging from about 70:1 to about 1:2, for example from about 30:1 to about 1:1, for example from about 15:1 to about 2:1, for example from about 10:1 to about 4:1, and more for example is about 5:1.

### Steroid Alcohols or Esters Thereof

The LNPs as disclosed herein may comprise at least one a steroid alcohol (or sterol) or an ester thereof. The presence of sterol or an ester of sterol may improve structural stability of the lipid nanoparticles.

A sterol, or steroid alcohol, may be selected from the group consisting of cholesterol or its derivatives, ergosterol, desmosterol (3ß-hydroxy-5,24-cholestadiene), stigmasterol (stigmasta-5,22-dien-3-ol), lanosterol (8,24-lanostadien-3b-ol), 7-dehydrocholesterol (Δ5,7-cholesterol), dihydrolanosterol (24,25-dihydrolanosterol), zymosterol (5α-cholesta-8,24-dien-3ß-ol), lathosterol (5α-cholest-7-en-3ß-ol), diosgenin ((3β,25R)-spirost-5-en-3-ol), sitosterol (22,23-dihydrostigmasterol), sitostanol, campesterol (campest-5-en-3ß-ol), campestanol (5a-campestan-3b-ol), 24-methylene cholesterol (5,24(28)-cholestadien-24-methylen-3ß-ol); BHEM-Cholesterol (2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]34henanthrene-3-yl)oxy)carbonyl)amino)-N,N-bis(2-hydroxyethyl)-N-methylethan-1-aminium bromide); and combinations thereof.

Esters of steroid alcohol or of sterol refer to ester of carboxylic acid with the hydroxyl group of the steroid alcohol. Suitable carboxylic acid comprises, further to the carboxyl moiety, a saturated or unsaturated, linear or branched, alkyl group. In some embodiments, the alkyl group may be a C₁-C₂₀ saturated or unsaturated, linear or branched, alkyl group, for example a C₂-C₁₈, for example a C₄-C₁₆, for example C₈-C₁₂ saturated or unsaturated, linear or branched, alkyl group. In other embodiments, the carboxylic acid may be a fatty acid. For example, a fatty acid may be caprylic acid, capric acid, lauric acid, stearic acid, margaric acid, oleic acid, linoleic acid, or arachidic acid.

In one embodiment, an ester of sterol may be a cholesteryl ester.

Esters of sterol or of steroid alcohol may be selected from the group consisting of cholesteryl margarate (cholest-5-en-3ß-yl heptadecanoate), cholesteryl oleate, cholesteryl stearate; and combinations thereof.

Sterols or steroid alcohols or esters thereof may be selected from selected from the group consisting of cholesterol or its derivatives, ergosterol, desmosterol (3ß-hydroxy-5,24-cholestadiene), stigmasterol (stigmasta-5,22-dien-3-ol), lanosterol (8,24-lanostadien-3b-ol), 7-dehydrocholesterol (Δ5,7-cholesterol), dihydrolanosterol (24,25-dihydrolanosterol), zymosterol (5α-cholesta-8,24-dien-3ß-ol), lathosterol (5α-cholest-7-en-3ß-ol), diosgenin ((3β,25R)-spirost-5-en-3-ol), sitosterol (22,23-dihydrostigmasterol), sitostanol, campesterol (campest-5-en-3ß-ol), campestanol (5a-campestan-3b-ol), 24-methylene cholesterol (5,24(28)-cholestadien-24-methylen-3ß-ol), cholesteryl margarate (cholest-5-en-3ß-yl heptadecanoate), cholesteryl oleate, cholesteryl stearate, and combinations thereof.

Alternatively, a sterol may be a cholesterol derivative such as an oxidized cholesterol.

Oxidized cholesterols suitable for the disclosure may be 25-hydroxycholesterol, 27-hydroxycholesterol, 20α-hydroxycholesterol, 6-keto-5α-hydroxycholesterol, 7-keto-cholesterol, 7β,25-hydroxycholesterol, 7β-hydroxycholesterol; and combinations thereof. For example, oxidized cholesterols may be 25-hydroxycholesterol and 20α-hydroxycholesterol, and for example it may be 20α-hydroxycholesterol.

In one embodiment, a sterol or steroid alcohol, or ester thereof may be cholesterol, a cholesteryl ester, or a cholesterol derivative, for example an oxidized cholesterol. In one embodiment, a sterol or steroid alcohol may be cholesterol or a cholesteryl ester, and for example may be cholesterol.

In one embodiment, a sterol or steroid alcohol may be cholesterol.

The LNPs may comprise from about 20 to about 55%, or from about 20% to about 50%, or from about 25% to about 45%, of said steroid alcohol, or ester thereof, in % w/w relative to the total weight of the lipid components of said LNPs.

In one embodiment, a sterol or steroid alcohol may be cholesterol, for example present in amount of about 28.5%, or about 38.5%, or about 40.9%, or about 42.7%, in % w/w relative to the total weight of the lipid components of said LNPs.

Sterols or steroid alcohols, or esters thereof, may be present in LNPs in a molar ratio ionizable cationic lipid:steroid alcohol, or ester thereof, ranging from about 4:1 to about 1:2, for example from about 3.5:1 to about 1:1.8, for example from about 2:1 to about 1:1.5, for example from about 1.5:1 to about 1:1.2, and for example is about 1.3:1 to about 1:1.3.

### PEG-Lipids

Lipid nanoparticles may include a PEG-lipid (or PEGylated lipid).

Contemplated PEG-modified lipids include, but are not limited to, a polyethylene glycol chain of up to 5 kDa in length covalently attached to a lipid with alkyl chain(s) of C₆-C₂₀ length. The addition of PEG-modified lipids to a composition of LNPs may prevent complex aggregation and may also provide a means for increasing circulation lifetime and increasing the delivery of the composition or lipid nanoparticles to the target cells.

A suitable PEGylated lipid may be, for example, a pegylated diacylglycerol (PEG-DAG), such as λ-(monomethoxy-polyethyleneglycol)-2,3-dimyristoylglycerol (PEG-DMG); a pegylated phosphatidylethanoloamine (PEG-PE); a PEG succinate diacylglycerol (PEG-S-DAG) such as 4-O-(2′,3′-di(tetradecanoyloxy)propyl-λ-0-(co-methoxy(polyethoxy) ethyl)butanedioate (PEG-S-DMG); a pegylated ceramide (PEG-cer); a PEG dialkoxypropylcarbamate, such as ω-methoxy(polyethoxy)ethyl-N-(2,3-di(tetradecanoxy) propyl)carbamate; 2,3-di(tetradecanoxy)propyl-N-(co-methoxy(polyethoxy)ethyl) carbamate; 2-[(polyethylene glycol)-2000]-N,N-ditetradecylacetamide (ALC-0159); and combinations thereof.

In one embodiment, a suitable PEGylated lipid may be selected from the group consisting of PEG-DAG; PEG-DMG; PEG-PE; PEG-S-DAG; PEG-S-DMG; PEG-cer; a PEG-dialkyoxypropylcarbamate; 2-[(polyethylene glycol)-2000]-N,N-ditetradecylacetamide (ALC-0159); and combinations thereof.

For example, a PEGylated lipid may be PEG-DMG PEG-PE, or 2-[(polyethylene glycol)-2000]-N,N-ditetradecylacetamide (ALC-0159).

In one embodiment, a PEG-lipid may be a PEG-PE, such as a PEG-2000-PE.

In one embodiment, a PEG-lipid may be a PEG-DMG, such as a DMG-PEG-2000.

In one embodiment, a PEG-lipid may be 2-[(polyethylene glycol)-2000]-N,N-ditetradecylacetamide (ALC-0159).

LNPs may comprise PEG-lipid in a molar amount ranging from about 1 to 30 about 15%, for example from about 1% to about 10%, for example from about 1% to about 5%, and for example from about 1% to about 3.5% relative to the total molar amount of the lipid components of the LNPs.

In one embodiment, a PEG-lipid may be DMG-PEG-2000, for example present in amount of about 1.5%, in % w/w relative to the total weight of the lipid components of said LNPs.

In one embodiment, a PEG-lipid may be 2-[(polyethylene glycol)-2000]-N,N-ditetradecylacetamide (ALC-0159), for example present in amount of about 1.6% or about 1.7%, in % w/w relative to the total weight of the lipid components of said LNPs.

PEG-lipid and the ionizable cationic lipid may be present in LNPs in a molar ratio ionizable cationic lipid to PEG-lipid from about 70:1 to about 4:1, for example from about 40:1 to about 10:1, for example from about 35:1 to about 15:1, and for example is about 33:1 or about 14:1.

In one embodiment, the LNPs may comprise an ionizable cationic lipid, a neutral lipid, a steroid alcohol or an ester thereof, and a PEG-lipid in a molar amount of about 20% to about 60% of ionizable cationic lipid, of about 5% to about 50% of neutral lipid, of 20% to about 55% of steroid alcohol or an ester thereof, and of about 0.5% to about 15% of PEG-lipid, in % w/w relative to the total weight of the lipid components of said LNPs.

In one embodiment, the LNPs may comprise an ionizable cationic lipid, a neutral lipid, a steroid alcohol or an ester thereof, and a PEG-lipid in a molar amount of about 35% to about 55% of ionizable cationic lipid, of about 5% to about 35% of neutral lipid, of about 25% to about 45% of steroid alcohol or an ester thereof, and of about 1.0% to about 2.5% of PEG-lipid, in % w/w relative to the total weight of the lipid components of said LNPs.

In one embodiment, the LNPs may comprise an ionizable cationic lipid, a neutral lipid, a steroid alcohol or an ester thereof, and a PEG-lipid in a molar amount of about 40% to about 50% of ionizable cationic lipid, of about 9% to about 30% of neutral lipid, of about 28% to about 45% of steroid alcohol or an ester thereof, and of about 1.5% to about 2.5% of PEG-lipid, in % w/w relative to the total weight of the lipid components of said LNPs.

In one embodiment, the molar ratio of the ionizable cationic lipid and of the neutral lipid, the steroid alcohol or an ester thereof, and the PEG-lipid may be of about 35/16/46.5/1.5, of about 50/10/38.5/1.5, of about 57.2/7.1/34.3/1.4, of about 40/15/40/5, of about 50/10/35/4.5/0.5, of about 50/10/35/5, of about 40/10/40/10, of about 35/15/40/10, or of about 52/13/30/5.

In one embodiment, the molar ratio of the ionizable cationic lipid and of the neutral lipid, the steroid alcohol or an ester thereof, and the PEG-lipid may be of about 35/16/46.5/1.5 or about 50/10/38.5/1.5.

In one embodiment, the LNPs may comprise an ionizable cationic lipid, a neutral lipid, a steroid alcohol or an ester thereof, and a PEG-lipid in a molar amount of about 50% of ionizable cationic lipid, of about 10% of neutral lipid, of about 38.5% of steroid alcohol or an ester thereof, and of about 1.5% of PEG-lipid, in % w/w relative to the total weight of the lipid components of said LNPs.

In one embodiment, the LNPs may comprise an ionizable cationic lipid, a neutral lipid, a steroid alcohol or an ester thereof, and a PEG-lipid in a molar amount of about 46.3% of ionizable cationic lipid, of about 9.4% of neutral lipid, of about 42.7% of steroid alcohol or an ester thereof, and of about 1.6% of PEG-lipid, in % w/w relative to the total weight of the lipid components of said LNPs.

In one embodiment, the LNPs may comprise an ionizable cationic lipid, a neutral lipid, a steroid alcohol or an ester thereof, and a PEG-lipid in a molar amount of about 40% of ionizable cationic lipid, of about 30% of neutral lipid, of about 28.5% of steroid alcohol or an ester thereof, and of about 1.5% of PEG-lipid, in % w/w relative to the total weight of the lipid components of said LNPs.

In one embodiment, the ionizable cationic lipid may be Dlin-MC3-DMA (or (6Z,9Z,28Z,31Z)-heptatriacont-6,9,28,31-tetraene-19-yl 4-(dimethylamino)butanoate, 9-heptadecanyl 8-{(2-hydroxyethyl)[6-oxo-6-(undecyloxy)hexyl]amino}octanoate (SM-102), or [(4-hydroxybutyl)azanediyl]di(hexane-6,1-diyl) bis(2-hexyldecanoate) (ALC-0315);

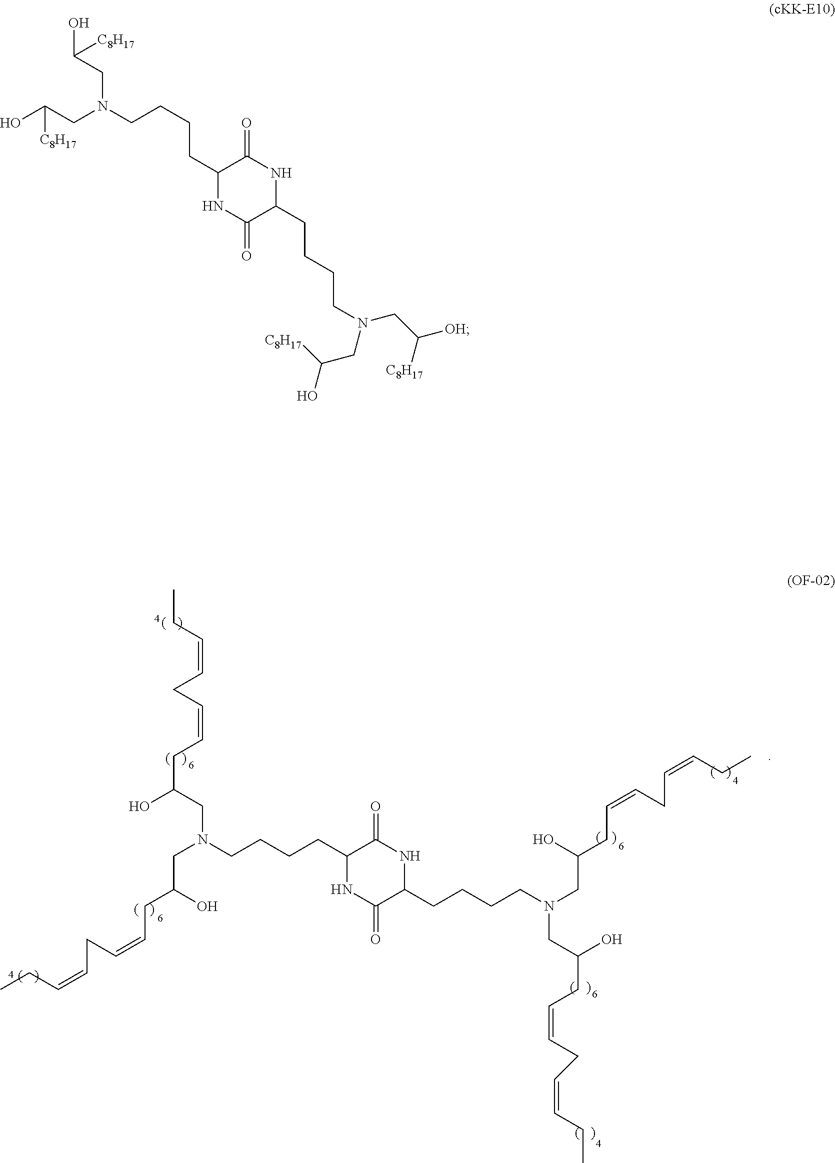

In one embodiment, the neutral lipid may be DSPC or DOPE.

In one embodiment, the steroid alcohol may be cholesterol.

In one embodiment, PEG-lipid may be PEG-PE (PEG-2000-PE) or PEG-DMG (PEG-2000-DMG).

In one embodiment, the ionizable cationic lipid may be Dlin-MC3-DMA, the neutral lipid may be DSPC, the steroid alcohol may be cholesterol, and the PEG-lipid may be PEG-DMG (DMG-PEG-2000).

In one embodiment, the LNPs may comprise 50% of Dlin-MC3-DMA, 10% of DSPC, 38.5% of cholesterol, and 1.5% of PEG-DMG (PEG-2000-DMG), in % w/w relative to the total weight of the lipid components of said LNPs.

In one embodiment, the ionizable cationic lipid may be 9-heptadecanyl 8-{(2-hydroxyethyl)[6-oxo-6-(undecyloxy)hexyl]amino}octanoate (SM-102), the neutral lipid may be DSPC, the steroid alcohol may be cholesterol, and the PEG-lipid may be PEG-DMG (DMG-PEG-2000).

In one embodiment, the LNPs may comprise 50% of 9-heptadecanyl 8-{(2-hydroxyethyl)[6-oxo-6-(undecyloxy)hexyl]amino}octanoate (SM-102), 10% of DSPC, 38.5% of cholesterol, and 1.5% of DMG-PEG-2000, in % w/w relative to the total weight of the lipid components of said LNPs.

In one embodiment, the ionizable cationic lipid may be [(4-hydroxybutyl)azanediyl]di(hexane-6,1-diyl) bis(2-hexyldecanoate) (ALC-0315), the neutral lipid may be DSPC, the steroid alcohol may be cholesterol, and the PEG-lipid may be PEG-DMG (DMG-PEG-2000).

In one embodiment, the LNPs may comprise 46.3% of [(4-hydroxybutyl)azanediyl]di(hexane-6,1-diyl) bis(2-hexyldecanoate) (ALC-0315), 9.4% of DSPC, 42.7% of cholesterol, and 1.6% of 2-[(polyethylene glycol)-2000]-N,N-ditetradecylacetamide (ALC-0159), in % w/w relative to the total weight of the lipid components of said LNPs.

In one embodiment, the LNPs may comprise 47.4% of [(4-hydroxybutyl)azanediyl]di(hexane-6,1-diyl) bis(2-hexyldecanoate) (ALC-0315), 10% of DSPC, 40.9% of cholesterol, and 1.7% of 2-[(polyethylene glycol)-2000]-N,N-ditetradecylacetamide (ALC-0159), in % w/w relative to the total weight of the lipid components of said LNPs.

In one embodiment, the ionizable cationic lipid may be cKK-E10, the neutral lipid may be DOPE, the steroid alcohol may be cholesterol, and the PEG-lipid may be PEG-DMG (DMG-PEG-2000).

In one embodiment, the LNPs may comprise 40% of cKK-E10, 30% of DOPE, 28.5% of cholesterol, and 1.5% of DMG-PEG-2000, in % w/w relative to the total weight of the lipid components of said LNPs.

In one embodiment, the ionizable cationic lipid may be ML7/OF-02, the neutral lipid may be DOPE, the steroid alcohol may be cholesterol, and the PEG-lipid may be PEG-DMG (DMG-PEG-2000).

In one embodiment, the LNPs may comprise 40% of ML7/OF-02, 30% of DOPE, 28.5% of cholesterol, and 1.5% of DMG-PEG-2000, in % w/w relative to the total weight of the lipid components of said LNPs.

### Lipid Nanoparticles (LNPs)

The lipid nanoparticles (LNPs) may be characterized by several parameters well-known in the art, such as the mean diameter size, the mode diameter size, the polydispersity index (PI) which reflects the homogeneity of the size distribution of the LNPs, the pKa, and/or the zeta potential which reflects the global surface charge of the LNPs.

The LNPs may be used to encapsulate at least one therapeutic agent. The encapsulation rate and the total content of such agent may also be used as parameters characterizing the LNPs.

Mode diameter size, mean diameter size and PI may be measured by Nanoparticles Tracking Analysis (NTA) NS300 from Malvern equipped with a 96 well plate auto-sampler or dynamic light scattering (DLS). pKa may be determined using a fluorescent probe 2-(p-toluidino)-6-napthalene sulfonic acid (TNS). Zeta potential can be determined using electrophoretic mobility or dynamic electrophoretic mobility measurements, for example with the Nicomp 380 ZLS system or the Malvern nanoZS.

The “mean diameter size” of the LNPs may be determined by Nanoparticles Tracking Analysis (NTA), and represents the average diameter of all particles analyzed in the sample. The “mode diameter size” represents the size of the most frequent particles population in number of the sample. Otherwise said, it is the size of the particles with the highest frequency. In regards of the size distribution profile of the sample, the mode diameter size represents the highest point of the peak seen in the distribution.

NTA uses the properties of both Brownian motion and light scattering for obtaining the particle size distribution of samples in a liquid suspension. A laser beam is passed through the sample chamber and the particles in suspension in the beam path scatter light such that they can be seen through a magnification microscope onto which a camera is mounted. The particle movement is captured on a frame-by-frame basis. The center of each of the observed particles is identified and tracked to obtain the average distance moved in the x and y planes. This value helps determine the particle diffusion coefficient (Dt) from which by knowing the sample temperature T and the solvent viscosity η, the sphere-equivalent hydrodynamic diameter d of the particles is determined with the Stokes-Einstein equation:

$$D t = \frac{T {K}_{B}}{3 πη d}$$

where KB is Boltzmann's constant.

The LNPs may have a diameter making them suitable for systemic, for example parenteral, or for intramuscular, intradermic, or subcutaneous administration. Typically, the lipid nanoparticles have a mean average diameter size of less than 600 nanometers (nm), for example of less than 400 nm.

In one embodiment, the LNPs have a mean average diameter size of less than 200 nm. Such size is advantageously compatible with sterile filtration and most appropriate for migration through the lymphatic vessels after intramuscular or subcutaneous administration. This size is also appropriate for intravenous administration, since larger particle injection could induce capillary thrombosis.

In some embodiments, the LNPs may have a mean diameter size in the range of from about 20 nm to about 300 nm, for example from about 25 nm to about 250 nm, for example from about 30 nm to about 200 nm, from about 40 nm to about 180 nm, from about 60 nm to about 170 nm, from about 70 to about 160 nm, and from about 80 to about 150 nm. In one embodiment, the LNPs may have a mean diameter size in the range of about 85 to about 140 nm, as measured by NTA. In a liquid composition (step a) of a method disclosed herein), the LNPs may have a mode diameter size from about 70 nm to about 250 nm, or from about 80 nm to about 200 nm, or from about 85 to about 140 nm, or of about 90 to about 120 nm, as measured by NTA.

The NTA technique requires that the sample is liquid. Therefore, for the determination of the mean diameter size of the LNPs after the freezing or the freeze-drying step, the obtained frozen or freeze-dried LNPs are thawed or resuspended in a solution, such as an aqueous buffer or water for injection (WFI).

The freezing methods and the spray-freeze-drying as disclosed herein may have no or reduced effect on the mode diameter size of LNPs comprising at least, as lipid components, a cationic ionizable lipid, a neutral lipid, and a steroid alcohol, or an ester thereof. Also, the freezing methods and the spray-freeze-drying as disclosed herein may have no or reduced effect on the mode diameter size of LNPs comprising at least, as lipid components, a cationic ionizable lipid, a neutral lipid, a steroid alcohol, or an ester thereof, and a PEG-lipid. The stability of the LNPs is therefore no or minimally affected by the methods as disclosed herein.

The stability of LNPs may be assessed by measuring the values of some parameters characterizing the LNPs, before and after application of a method as disclosed herein or after application of a method as disclosed herein and along a certain period of time.

Parameters of LNPs which may be measured for assessing LNPs' stability may be, for example, mode diameter size as measure by NTA or encapsulation rate of agents, such as mRNA, possibly loaded in the LNPs.

For example, the mode diameter size of the LNPs may be measured for LNPs in the liquid composition before freezing, and for frozen LNPs. As indicated, above frozen LNPs have to be thawed or resuspended in solution, for example with an aqueous buffer or water for injection, before being subjected to measures by NTA.

In some embodiments, the LNPs at the freezing step of a method disclosed herein may have a mode diameter size measured by NTA no greater than about 45%, or no greater than about 35%, or no greater than about 30%, or no greater than about 25%, or no greater than about 20%, or no greater than about 15% or no greater than about 10%, or no greater than about 8%, or no greater than about 5% of the mode diameter size of the LNPs in the liquid composition (before freezing).

A variation of the mode diameter size of the LNPs before and after the step of freezing of less than about 45%, or less than about 35%, or less than about 30%, or less than about 25%, or less than about 20%, or less than about 15% or less than about 10%, or less than about 8%, or less than about 5% may be indicative of a low or reduced aggregation effect during the freezing process.

The mode diameter size of the LNPs may also be measured for freeze-dried LNPs resuspended in solution, for example with an aqueous buffer or water for injection, before being subjected to measures by NTA

In some embodiments, the LNPs at the freeze-dried step of a method disclosed herein may have a mean diameter size measured by NTA no greater than about 15%, or no greater than about 13%, or no greater than about 11%, or no greater than about 10%, or no greater than about 5%, of the mean diameter size of the LNPs in the liquid composition.

A variation of the mode diameter size of the LNPs before and after the step of spray-freeze drying of less than about 15%, or less than about 13%, or less than about 11%, or less than about 10%, or less than about 5%, may be indicative of a low or reduced aggregation effect during the spray-freeze drying process.

The impact of a spray-freeze drying method as disclosed herein may also be assessed overtime with regard to the freeze-dried LNPs. In such case, the freeze-dried LNPs may be stored at constant temperature, for example+5° C., and from time to time, e.g., every month, or 2-months, or 3-months, or 4-months, a sample of freeze-dried LNPs may resuspended in an aqueous buffer or water for injection for mode size diameter measure by NTA. The obtained measured may be then compared to a reference value, which may be the mode size diameter of the LNPs in liquid composition before freeze-drying or just after freeze-drying, i.e. at TO.

The lipid nanoparticles may comprise or encapsulate at least one therapeutic agent. Such agent may be encapsulated in and/or adsorbed on an exterior surface of the LNPs. Such agent may bear some positive or negative charges.

In case of LNPs containing a negatively charged therapeutic agent, e.g., a nucleic acid, the lipid nanoparticles may be formed by adjusting, for example at the time of the preparation, a positive (+) to negative (−) charge ratio of the ionizable cationic lipid (cationic charges) to the negatively charged agent (e.g., anionic charges from the phosphate in case of nucleic acid). The charges of the ionizable cationic lipid and of the negatively charged agent are charges at a selected pH, such as a physiological pH, which is from about 6.5 to about 7.5.

The +/−charge ratio of the ionizable cationic lipid to the negatively charged agent in the LNPs can be calculated by the following equation. (+/−charge ratio)=[(cationic lipid amount (mol))*(the total number of positive charges in the cationic lipid)]:[(negatively charged agent amount (mol))*(the total number of negative charges in negatively charged agent)].

The negatively charged agent amount and ionizable cationic lipid amount can be easily determined by one skilled in the art in view of a loading amount upon preparation of the LNPs.

According to an embodiment, the ratio of positive to negative charge in LNPs suitable for the present disclosure is such that they may have a global negative charge or a global charge at or near the neutrality.

In one embodiment, the charge ratio of positive charges to negative charges in the LNPs is ranging from about 4:1 to about 15:1, for example from about 5:1 to about 12:1, for example from about 6:1 to about 9:1, and for example is from about 6:1 to about 8:1.

In one embodiment, the charge ratio of positive charges to negative charges in the LNPs is about 6:1.

The present disclosure relates to frozen LNPs obtainable according to a method as disclosed herein.

The present disclosure relates to frozen LNPs comprising at least, as lipid components, a cationic ionizable lipid, a neutral lipid, and a steroid alcohol, or an ester thereof, the frozen LNPs being in frozen micropellets. Such frozen LNPs may further comprise a PEG-lipid. Such frozen LNPs further comprise a nucleic acid.

In one of its objects, the present invention relates to freeze-dried LNPs obtainable according to a method as disclosed herein

The present disclosure relates to freeze-dried LNPs comprising at least, as lipid components, a cationic ionizable lipid, a neutral lipid, and a steroid alcohol, or an ester thereof, said freeze-dried LNPs being in freeze-dried micropellets. Such freeze-dried LNPs may further comprise a PEG-lipid. Such freeze-dried LNPs further comprise a nucleic acid.

### Lipid Nanoparticles Manufacturing Process

Methods for manufacturing LNPs are known in the art.

In one embodiment, LNPs containing a therapeutic agent may be obtainable by a method comprising at least the steps of:
i) solubilizing, in a water miscible organic solvent, the lipid components of the LNPs,
ii) mixing the organic solvent obtained at step a) with an aqueous solvent containing a nucleic acid, and
iii) obtaining said LNPs, in the aqueous solvent.

In one embodiment, a method for manufacturing LNPs may comprise at least steps of:
i) solubilizing, in a water miscible organic solvent, at least one ionizable cationic lipid, at least neutral lipid, at least one steroid alcohol or ester thereof, and at least one PEG-lipid,
ii) mixing the organic solvent obtained at step a) with an aqueous solvent containing a nucleic acid, and
iii) obtaining the lipid nanoparticles containing a nucleic acid, in the aqueous solvent.

Useful water-miscible organic solvents may be any water-miscible organic solvent capable to solubilize the lipidic compound as disclosed herein and any other added lipids. As example of suitable organic solvents, one may cite ethanol or methanol, 1-propanol, isopropanol, t-butanol, THF, DMSO, acetone, acetonitrile, diglyme, DMF, 1-4 dioxane, ethylene glycol, glycerine, hexamethylphosphoramide, hexamethylphosphorous triamide. In one embodiment, the organic solvent may be ethanol and isopropanol.

Aqueous solvents usable at step ii) include aqueous buffered solutions.

As examples of suitable aqueous buffered solution, one may mention acidic buffer, such as include citrate buffer, sodium acetate buffer, succinate buffer, borate buffer or a phosphate buffer. For example, an aqueous buffered solvent may be a citrate buffered solution or an acetate buffered solution.

The pH of the aqueous solvent may range from about 3.5 to about 7.0, for example from about 4.0 to about 6.5, and for example from about 4.5 to about 6.0, and for example may be at about 5.5. In one embodiment, the pH may be of about 4.0.

At step ii), the organic and aqueous solvents may be mixed at a ratio organic solvent:aqueous solvent ranging from about 1:1 to about 1:6. In one embodiment, the ratio may range from about 1:2 to about 1:4, and for example may be a ratio of about 1:3.

According to one embodiment, the organic solvent and the aqueous solvent may be mixed at step b) at a flow rate ranging from about 0.01 ml/min to about 12 ml/min. In some embodiments, the flow rate may range from about 0.02 ml/min to about 10 ml/min, from about 0.5 ml/min to about 8 ml/min, from about 1 ml/min to about 6 ml/min, or at about 4 ml/min.

The step of mixing may be carried by any known method in the art. For instance, both solvents may be mixed with a T-tube or a Y-connector. Alternatively, the mixing may be carried out by laminar flow mixing with a microfluidic micromixer as described by Belliveau et al. (Mol Ther Nucleic Acids. 2012; 1(8):e37), the content of which is incorporated by reference.

As indicated, the aqueous solvent at step b) comprises a nucleic acid. A suitable nucleic acid may be for example as detailed below.

The method may further comprise, if necessary, a step of increasing the pH from acidic to neutral.

In a further embodiment, the method may comprise a step iv) of increasing the pH of the aqueous solvent containing the LNPs obtained at step iii) at a pH ranging from about 5.5 to about 7.5, for example from about 6.0 to about 7.5.

The step of increasing the pH may be carried by any known method in the art. For example, the change in pH may carried by a dialyzing or diafiltration step.

Further, if needed, osmolarity may be adjusted to reach a final osmolality close to 290 mOsmol/kg as to inject isotonic solution into the body.

Further, a method for preparing LNPs may comprise any further step suitable to harvest, purify, concentrate and/or sterilize the lipid nanoparticles to further formulate them as a pharmaceutical composition, for example as an immunogenic composition.

### Formulations for Freezing and Freeze-Drying LNPs

Before being frozen or freeze-dried a composition comprising the LNPs may mixed with excipients. Such excipients may be a buffer solution, bulking agents, pH stabilizers, pH adjusters, thermal stabilizers, cryoprotectants, lyoprotectants, antioxidants,

The composition comprising the LNPs intended to be frozen or freeze-dried may be isotonic (isosmotic).

Such excipients, as cryoprotectants, may contribute to stabilize the LNPs during the freezing or the spray-freeze-drying methods.

### Cryoprotectants and Lyoprotectants

Liquid compositions containing LNPs may be added of at least one cryoprotectant.

The cryoprotectant or lyoprotectant may be selected from disaccharides (such as lactose, trehalose, sucrose, maltose, and mannose), sorbitol, amino acids, peptides, polymers and proteins such as albumins (bovine serum albumin, human serum albumin) or gelatins.

In some embodiments, a cryoprotectant may be a carbohydrate. In one embodiment, the cryoprotectant is a carbohydrate selected from a monosaccharide, a disaccharide, a trisaccharide, a sugar alcohol, an oligosaccharide or its corresponding sugar alcohol, and a straight chain polyalcohol. Exemplary disaccharide cryoprotectants include sucrose, trehalose, lactose, maltose and the like.

In one embodiment a cryoprotectant may be a polyol. In some embodiments, a cryoprotectant may be selected in a group consisting of mannose, sucrose, lactose, trehalose, maltose, sorbitol, mannitol, glycerol, inositol, glucose, fructose, arginin, glycerin, dextran, and mixtures thereof.

In some embodiments, a cryoprotectant may be trehalose. In some embodiments, a trehalose may be a trehalose dihydrate.

In some embodiments, a cryoprotectant may be dextran.

In some embodiments, a cryoprotectant is a mixture of trehalose and dextran.

In one embodiment, the trehalose is at a concentration from about 5 to about 50 weight by volume percent (% w/v), or from about 8% (w/v) to about 40% (w/v), or from about 10% (w/v) to about 25% (w/v), or from about 15% (w/v) to about 20% (w/v), relative to the total volume of the composition.

In one embodiment, the trehalose is at a concentration of about 16.25% (w/v).

Dextrans having a molecular mass of 1,000 to 100,000 Da and are preferred and more preferably 1,000 to 10,000 Da may be used. Dextrans may be used with other cryoprotectants.

In one embodiment, the dextran is at a concentration from about 5 to about 25 weight by volume percent (% w/v), or from about 8% (w/v) to about 20% (w/v), or from about 15% (w/v) to about 18% (w/v).

In one embodiment, the dextran is at a concentration of about 16.25% (w/v).

In one embodiment, the trehalose and the dextran are present in an equal amount of weight by volume percent, relative to the total volume of the composition.

In one embodiment, the cryoprotectant is a mixture of trehalose at a concentration of about 16.25% (w/v) and of dextran at a concentration of about 16.25% (w/v), relative to the total volume of the composition.

In some embodiments, a composition containing LNPs in accordance with the disclosure may comprise a cryoprotectant consisting essentially of trehalose and dextran.

In some embodiments, the disclosure relates to freeze-dried micropellets comprising LNPs comprising at least a nucleic acid and, at least, as lipid components, a cationic ionizable lipid, a neutral lipid, and a steroid alcohol, or an ester thereof and a cryoprotectant as above indicated. In some embodiments, a cryoprotectant is a mixture of trehalose and dextran.

### Buffer

The buffer may be selected from phosphate buffered saline, citrate buffer, Tris buffer, amino acid based buffers (such as histidine buffer, glycine buffer), sodium dihydrogen orthophosphate, disodium hydrogen orthophosphate, potassium dihydrogen orthophosphate, dipotassium hydrogen orthophosphate, TES, MOPS, PIPES, Cacodylate, SSC, MES and HEPES.

In some embodiments, a buffer may be a Tris-buffer.

In some embodiments, a buffer may be a phosphate buffered saline.

In some embodiments, a formulation does not comprise a buffer.

In some embodiments, a formulation does not comprise a Tris-buffer.

### Other Excipients

The composition comprising the LNPs and intended to be frozen or freeze-dried in methods as disclosed herein may further comprise additional excipient such as a thermal stabilizer, antioxidant, or a bulking agent.

The thermal stabilizer may be select from mannitol, polymers (such as dextran, polyethylene glycol, polyvinyl pyrrolidone) and proteins.

The antioxidant may be selected from: Vitamin A (retinol), Vitamin C (ascorbic acid) and Vitamin E (comprising tocotrienol and tocopherol).

The bulking agent may be selected from mannitol, polymers (such as dextran, polyethylene glycol, and polyvinyl pyrrolidone), disaccharides (such as lactose, trehalose, sucrose, maltose, and mannose), sorbitol and proteins such as albumins and gelatins.

### Nucleic Acids

A nucleic acid suitable for the present disclosure may be a deoxyribonucleic acid (DNA) or a ribonucleic acid (RNA). Nucleic acid includes genomic DNA, cDNA, mRNA, recombinantly produced and chemically synthesized molecules.

A nucleic acid may be a single-stranded or a double-stranded molecule, linear or closed covalently to form a circle. A nucleic acid may be a double stranded RNA (dsRNA); a single stranded RNA (ssRNA); a double stranded DNA (dsDNA); a single stranded DNA (ssDNA); and combinations thereof.

LNPs containing a nucleic may be employed for introduction into, i.e., transfection of, cells, of the nucleic acid, for example, for recombinant protein expression, for gene replacement, for suppressing or increasing expression of a host protein.

A nucleic acid may be of eukaryotic or prokaryotic origin, and for example of human, animal, plant, bacterial, yeast or viral origin and the like. It may be obtained by any technique known to persons skilled in the art and for example by screening libraries, by chemical synthesis or alternatively by mixed methods including chemical or enzymatic modification of sequences obtained by screening libraries. It may be chemically modified.

A nucleic acid may be comprised in a vector. Vectors are known to the skilled person and may include plasmid vectors, cosmid vectors, phage vectors such as lambda phage, viral vectors such as adenoviral or baculoviral vectors, or artificial chromosome vectors such as bacterial artificial chromosomes (BAC), yeast artificial chromosomes (YAC), or PI artificial chromosomes (PAC). Vectors include expression as well as cloning vectors. Expression vectors comprise plasmids as well as viral vectors and generally contain a desired coding sequence and appropriate DNA sequences necessary for the expression of the operably linked coding sequence in a specific host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments.

A nucleic acid may be a messenger RNA (mRNA); a microRNA (miRNA); a short (or small) interference RNA (siRNA); small hairpin RNA (shRNA); a long non-coding RNA (lncRNA); an asymmetrical interfering RNA (aiRNA); a self-amplifying RNA (saRNA); a small nuclear RNA (snRNA); a small nucleolar RNA (snoRNA); a guide RNA (gRNA); an anti-sense oligonucleotide (ASO); a plasmid DNA (pDNA); closed-ended DNA (ceDNA), and combinations thereof.

In some embodiments, a nucleic acid may be an RNA.

In some embodiments, a nucleic acid may be a messenger RNA (mRNA); a microRNA (miRNA); a short (or small) interference RNA (siRNA); small hairpin RNA (shRNA); a long non-coding RNA (lncRNA); an asymmetrical interfering RNA (aiRNA); a self-amplifying RNA (saRNA); a guide RNA (gRNA); and combinations thereof.

In some embodiments, LNPs may contain as nucleic acids an mRNA encoding for a CRISPR protein, such as CRISPR/Cas9, and a guide RNA (gRNA). A gRNA may be provided as rRNA:tracrRNA duplex or as a single guide RNA (sgRNA). In some embodiments, a CRISPR protein may be provided directly as a polypeptide and not as an mRNA encoding for a CRISPR protein.

In some embodiments, an RNA may be a messenger RNA (mRNA).

In some embodiments, a nucleic acid may encode a genome-editing polypeptide, a chemokine, a cytokine, a growth factor, an antibody, an enzyme, a structural protein, a blood protein, an hormone, a transcription factor, or an antigen, such as described herein.

Messenger RNA (mRNA)

mRNA is typically thought of as the type of RNA that carries information from DNA to the ribosome. The existence of mRNA is typically very brief and includes processing and translation, followed by degradation. Typically, in eukaryotic organisms, mRNA processing comprises the addition of a “cap” on the N-terminal (5′) end, and a “tail” on the C-terminal (3′) end.

A typical cap is a 7-methylguanosine cap, which is a guanosine that is linked through a 5′-5′-triphosphate bond to the first transcribed nucleotide. The presence of the cap is important in providing resistance to nucleases found in most eukaryotic cells. A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase.

The tail is typically a polyadenylation event whereby a polyadenylyl moiety is added to the 3′ end of the mRNA molecule. The presence of this “tail” serves to protect the mRNA from exonuclease degradation. Messenger RNA is translated by the ribosomes into a series of amino acids that make up a protein.

In some embodiments, mRNAs include a 5′ and/or 3′ untranslated region (UTR). In some embodiments, mRNA disclosed herein comprise a 5′ UTR that includes one or more elements that affect an mRNA's stability or translation. In some embodiments, a 5′ UTR may be between about 50 and 500 nucleotides in length. In some embodiments, mRNA disclosed herein comprise a 3′ UTR comprising one or more of a polyadenylation signal, a binding site for proteins that affect an mRNA's stability of location in a cell, or one or more binding sites for miRNAs. In some embodiments, a 3′ UTR may be between 50 and 500 nucleotides in length or longer. In some embodiments, the mRNAs disclosed herein comprise a 5′ or 3′ UTR that is derived from a gene distinct from the one encoded by the mRNA transcript. In some embodiments, the mRNAs disclosed herein comprise a 5′ or 3′ UTR that is chimeric.

The mRNAs disclosed herein may be synthesized according to any of a variety of known methods. For example, mRNAs according to the present invention may be synthesized via in vitro transcription (IVT). Methods for in vitro transcription are known in the art. See, e.g., Geall et al. (2013) Semin. Immunol. 25(2): 152-159; Brunelle et al. (2013) Methods Enzymol. 530:101-14, the content of which is incorporated by reference. Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor. The exact conditions will vary according to the specific application. The presence of these reagents is undesirable in a final mRNA product and are considered impurities or contaminants which must be purified to provide a clean and homogeneous mRNA that is suitable for therapeutic use. While mRNA provided from in vitro transcription reactions may be desirable in some embodiments, other sources of mRNA can be used according to the instant disclosure including wild-type mRNA produced from bacteria, fungi, plants, and/or animals.

The mRNA disclosed herein may be modified or unmodified. In some embodiments, the mRNA disclosed herein contain one or more modifications that typically enhance RNA stability. Exemplary modifications include backbone modifications, sugar modifications, or base modifications. In some embodiments, the disclosed mRNAs may be synthesized from naturally occurring nucleotides and/or nucleotide analogues (modified nucleotides) including, but not limited to, purines (adenine (A), guanine (G)) or pyrimidines (thymine (T), cytosine (C), uracil (U)), and as modified nucleotides analogues or derivatives of purines and pyrimidines, such as e.g. 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxy acetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, β-D-mannosyl-queosine, phosphoramidates, phosphorothioates, peptide nucleotides, methylphosphonates, 7-deazaguanosine, 5-methylcytosine, and inosine. In some embodiments, the disclosed mRNAs comprise at least one chemical modification including but not limited to, consisting of pseudouridine, N1-methylpseudouridine, 2-thiouridine, 4′-thiouridine, 5-methylcytosine, 2-thio-1-methyl-1-deaza-pseudouridine, 2-thio-I-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine. In some embodiments, the modified nucleotides comprise N1-methylpseudouridine. The preparation of such analogues is known to a person skilled in the art e.g., from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530, and 5,700,642, the content of which is incorporated by reference.

The term “RNA” relates to a molecule which comprises ribonucleotide residues and for example being entirely or substantially composed of ribonucleotide residues. “Ribonucleotide” relates to a nucleotide with a hydroxyl group at the 2′-position of a β-D-ribofuranosyl group. It includes double stranded RNA, single stranded RNA, isolated RNA such as partially purified RNA, essentially pure RNA, synthetic RNA, or recombinantly produced RNA.

For the sake of clarity, a mRNA encompasses any coding RNA molecule, which may be translated by a eukaryotic host into a protein. A coding RNA molecule generally refers to an RNA molecule comprising a sequence coding for a protein of interest, and which may be translated by the eukaryotic host, said sequence starting with a start codon (ATG) and for example terminated by a stop codon (i.e. TAA, TAG. TGA).

An RNA may be a naturally occurring RNA or a modified RNA that differs from naturally occurring RNA by the addition, deletion, substitution and/or alteration of at least one nucleotide. Such alterations can include addition of non-nucleotide material, such as to the end(s) of a RNA or internally, for example at least one nucleotide of the RNA. Nucleotides in RNA molecules can also comprise non-standard nucleotides, such as non-naturally occurring nucleotides or chemically synthesized nucleotides or deoxynucleotides. These altered RNAs can be referred to as analogs or analogs of naturally occurring RNA.

A mRNA may be produced by in vitro transcription using a DNA template. Alternatively, the RNA may be obtained by chemical synthesis. Such methods are known to the skilled person. For example, there is a variety of in vitro transcription kits commercially available.

An RNA may be in vitro synthesized in a cell-free system, using appropriate cell extracts and an appropriate DNA template. For example, cloning vectors are applied for the generation of transcripts. The promoter for controlling transcription can be any promoter for any RNA polymerase. Some examples of RNA polymerases are the T7, T3, and SP6 RNA polymerases. A DNA template for in vitro transcription may be obtained by cloning of a nucleic acid, for example a cDNA, and introducing it into an appropriate vector for in vitro transcription. The cDNA may be obtained by reverse transcription of RNA. For example, cloning vectors are used for producing transcripts which generally are designated transcription vectors.

An RNA may encode for a protein or a peptide. That is, if present in the appropriate environment, for example within a cell, such as an antigen-presenting cell, for example a dendritic cell, the RNA can be expressed to produce a protein or peptide it encodes. The stability and translation efficiency of an RNA may be modified as required.

In some embodiments, an mRNA may encode a genome-editing polypeptide, a chemokine, a cytokine, a growth factor, an antibody, an enzyme, a structural protein, a blood protein, an hormone, a transcription factor, or an antigen, such as described herein.

In some embodiments, an mRNA may encode for an antigen.

RNA molecules may be of variable length. Thus, they may be short RNA molecules, for instance RNA molecules shorter than about 100 nucleotides, or long RNA molecules, for instance longer than about 100 nucleotides, or even longer than about 300 nucleotides.

An mRNA may be at least 30 nucleotides in length.

An mRNA may comprise a 5′Cap structure, a 5′-UTR sequence, an ORF sequence coding for a protein or a peptide, a 3′-UTR sequence, and a poly(A) tail.

Typically, a mRNA may comprise or consist of the following general formula:

[5′Cap]w—[5′UTR]x—[Gene of Interest]—[3′UTR]y—[PolyA]z
wherein [5′Cap] contains a methyl guanine nucleotide linked to mRNA via a 5′ to 5′ linkage,
wherein [5′UTR] and [3′UTR] are untranslated regions (UTR),
wherein [5′UTR] contains a Kozak sequence,
wherein [Gene of Interest] is any gene coding for a protein of interest,
wherein [PolyA] is a poly(A) tail, and
wherein w, x, y, and z, are identical or different, and equal to 0 or 1.

A Kozak sequence refers to a sequence, which is generally a consensus sequence, occurring in eukaryotic mRNAs and which plays a major role in the initiation of the translation process. Kozak sequences and Kozak consensus sequences are well known in the art.

The [3′UTR] does not express any proteins. The purpose of the [3′UTR] is to increase the stability of the mRNA. According to a one embodiment, the α-globin UTR is chosen because it is known to be devoid of instability.

A sequence corresponding to the gene of interest may be codon-optimized in order to obtain a satisfactory protein production within the host which is considered.

A poly(A) tail consists of multiple adenosine monophosphates that is well known in the art. A poly(A) tail is generally produced during a step called polyadenylation that is one of the post-translation modifications which generally occur during the production of mature messenger RNAs. Such poly(A) tail contributes to the stability and the half-life of the mRNA, and can be of variable length. For example, a poly(A) tail may be equal or longer than 10 A nucleotides, which includes equal or longer than 20 A nucleotides, which includes equal or longer than 100 A nucleotides, and for example about 120 A nucleotides.

An RNA molecule may encompass:
(i) capped unmodified RNA molecule;
(ii) capped modified RNA molecule;
(iii) uncapped unmodified RNA molecule;
(iv) uncapped modified RNA molecule.

### Capped and Uncapped RNA Molecules

A “capped RNA molecule” refers to an RNA molecule of which the 5′end is linked to a guanosine or a modified guanosine, for example a 7-methylguanosine (m⁷G), connected to a 5′ to 5′ triphosphate linkage or analog. This definition is commensurate with the most widely-accepted definition of a 5′cap.

“Cap analogs” include caps which are biologically equivalent to a 7-methylguanosine (m⁷G), connected to a 5′ to 5′ triphosphate linkage, and which can thus be also substituted without impairing the protein expression of the corresponding messenger RNA in the eukaryotic host.

As example of caps, one may mention m⁷GpppN, m⁷GpppG, m⁷GppspG, m⁷GppspspG, m⁷GppspspG, m⁷Gppppm7G, m₂^{7′,3′}-OGpppG, m₂^{7′,2′}-OGpppG, m₂^{7′,2′}-OGppspsG, or m₂^{7′,2′}-OGpppspsG.

Examples of cap analogs can be: glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranos I nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety.

Other examples of cap analogs include Anti-Reverse Cap Analogs (ARCAs), N¹-methyl-guanosine, 2′-fluoro-guanosine, 7-deaza-guanosine, 8-oxo-guanosine, 2-amino-guanosine, LNA-guanosine, and 2-azido-guanosine.

Of note, among cap analogs, some are suitable for protein expression, but others may on the contrary hinder protein expression. Such distinction is understood by the man skilled in the art.

Providing a RNA with a 5′-cap or 5′-cap analog may be achieved by in vitro transcription of a DNA template in the presence of said 5′-cap or 5′-cap analog, wherein said 5′-cap is co-transcriptionally incorporated into the generated RNA strand, or the RNA may be generated, for example, by in vitro transcription, and the 5′-cap may be attached to the RNA post-transcriptionally using capping enzymes, for example, capping enzymes of vaccinia virus.

An “uncapped RNA molecule” refers to any RNA molecule that does not belong to the definition of a “capped RNA molecule”.

Thus, according to a general embodiment, an “uncapped mRNA” may refer to a mRNA of which the 5′end is not linked to a 7-methylguanosine, through a 5′ to 5′ triphosphate linkage, or an analog as previously defined.

An uncapped RNA molecule, such as a messenger RNA, may be an uncapped RNA molecule having a (5′)ρρρ(5′), a (5′)ρρ(5′), a (5′)ρ(5′) or even a (5′)OH extremity. Such RNA molecules may be respectively abbreviated as 5′ρρρRNA; 5′ρρRNA; 5′ρRNA; 5′OHRNA.

In a non-limitative manner, the first base of an uncapped RNA molecule may be either an adenosine, a guanosine, a cytosine, or an uridine.

An RNA may not have uncapped 5′-triphosphates. Removal of such uncapped 5′-triphosphates can be achieved by treating RNA with a phosphatase.

### Modified and Unmodified RNA Molecules

An RNA may comprise further modifications, such as an extension or a truncation of the naturally occurring poly(A) tail or an alteration of the 5′- or 3′-untranslated regions (UTR) such as introduction of an UTR which is not related to the coding region of the RNA, for example, the exchange of the existing 3′-UTR with or the insertion of at least one, for example two copies of a 3′-UTR derived from a globin gene, such as alpha 2-globin, alpha 1-globin, beta-globin, for example beta-globin, and for example human beta-globin.

A “modified RNA molecule” refers to an RNA molecule which contains at least one modified nucleotide, nucleoside sugar, or base, such as a modified purine or a modified pyrimidine. A modified nucleoside or base can be any nucleoside or base that is not A, U, C or G (respectively Adenosine, Uridine, Cytidine or Guanosine for nucleosides; and Adenine, Uracil, Cytosine or Guanine when referring solely to the sugar moiety).

An “unmodified RNA molecule” refers to any RNA molecule that is not commensurate with the definition of a modified RNA molecule.

The terms “modified and unmodified” are considered distinctly from the terms “capped and uncapped”, as the latter specifically relates to the base at the 5′-end of a RNA.

The presence of modified nucleotide may increase the stability and/or decrease cytotoxicity of the nucleic acid. The term stability of RNA relates to the half-life of RNA, that is the period of time which is needed to eliminate half of the activity, amount, or number of molecules. The half-life of an RNA may be indicative of its stability. The half-life of RNA may influence the duration of expression of the RNA. It can be expected that an RNA having a long half-life will be expressed for an extended time period.

In a non-limitative manner, examples of modified nucleotides, nucleosides and bases are disclosed in WO 2015/024667A1. A modified RNA may contain modified nucleotides, nucleosides or bases, including backbone modifications, sugar modifications or base modifications. Modified bases and/or modified RNA molecules are known in the art and are, for instance, taught in Warren et al. (“Highly Efficient Reprogramming to Pluripotency and Directed Differentiation of Human Cells with Synthetic Modified mRNA”; Cell Stem Cell; 2010), the content of which is incorporated by reference.

Sugar modifications include chemical modifications of the sugar of the nucleotides. Sugar modifications may consist in replacement or modification of the 2′ hydroxy (OH) group, which can be modified or replaced with a number of different “oxy” or “deoxy” substituents.

Examples of “oxy”-2′ hydroxyl group modifications include, but are not limited to, alkoxy or aryloxy (—OR, e.g., R═H, alkyl, cycloalkyl, aryl, aralkyl, heteroaryl or sugar); polyethyleneglycols (PEG), —O(CH2CH2O)nCH2CH2OR; “locked” nucleic acids (LNA) in which the 2′ hydroxyl is connected, e.g., by a methylene bridge, to the 4′ carbon of the same ribose sugar; and amino groups (—O-amino, wherein the amino group, e.g., NRR, can be alkylamino, dialkylamino, heterocyclyl, arylamino, diarylamino, heteroarylamino, or diheteroaryl amino, ethylene diamine, polyamino) or aminoalkoxy.

“Deoxy” modifications include hydrogen, amino (e.g., NH2; alkylamino, dialkylamino, heterocyclyl, arylamino, diaryl amino, heteroaryl amino, diheteroaryl amino, or amino acid); or the amino group can be attached to the sugar through a linker, wherein the linker comprises at least one of the atoms C, N, and O.

The sugar group can also contain at least one carbon that possess the opposite stereochemical configuration than that of the corresponding carbon in ribose. Thus, a modified RNA can include nucleotides containing, for instance, arabinose as the sugar.

Backbone modifications include modifications, in which phosphates of the backbone of the nucleotides are chemically modified. The phosphate groups of the backbone can be modified by replacing at least one of the oxygen atoms with a different substituent. Further, the modified nucleosides and nucleotides can include the full replacement of an unmodified phosphate moiety with a modified phosphate as described herein.

Examples of modified phosphate groups include, but are not limited to, phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. The phosphate linker can also be modified by the replacement of a linking oxygen with nitrogen (bridged phosphoroamidates), sulfur (bridged phosphorothioates) and carbon (bridged methylene-phosphonates).

Base modifications include chemical modifications of the base moiety of the nucleotides. In this context nucleotide analogues or modifications are for example selected from nucleotide analogues which are suitable for transcription and/or translation of the RNA molecule in an eukaryotic cell. The modified nucleosides and nucleotides can be modified in the nucleobase moiety. For example, the nucleosides and nucleotides can be chemically modified on the major groove face. The major groove chemical modifications can include an amino group, a thiol group, an alkyl group, or a halo group. A modified base may be a modified purine base or a modified pyrimidine base. Examples of modified purine bases include modified adenosine and/or modified guanosine, such as hypoxanthine; xanthine; 7-methylguanine; inosine; xanthosine and 7-methylguanosine. Modified pyrimidine bases include modified cytidine and/or modified uridine, such as 5,6-dihydrouracil; pseudouridine; 5-methylcytidine; 5-hydroxymethylcytidine; dihydrouridine and 5-methylcytidine.

For examples, nucleotide analogues/modifications may be selected from the following base modifications: 2-amino-6-chloropurineriboside-5′-triphosphate, 2-aminopurine-riboside-5′-triphosphate; 2-aminoadenosine-5′-triphosphate, 2′-amino-2′-deoxycytidine-triphosphate, 2-thiocytidine-5′-triphosphate, 2-thiouridine-5′-triphosphate, 2′-fluorothymidine-5′-triphosphate, 2′-O-methyl inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5′-triphosphate, 5-methyluridine-5′-triphosphate, 5-propynyl-2′-deoxycytidine-5′-triphosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, O6-methylguanosine-5′-triphosphate, pseudouridine-5′-triphosphate, or puromycin-5′-triphosphate, and xanthosine-5′-triphosphate.

Modified nucleosides may be selected from a list consisting of: pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, I-taurinomethyl-4-thio-uridine, 5-methyl-uridine, 1-methyl-pseudouridine, 4-thio-1-methyl-pseudouridine, 2-thio-I-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine/4-methoxy-pseudouridine, and 4-methoxy-2-thio-pseudouridine.

Modified nucleosides and nucleotides may include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

Modified nucleosides may include 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N⁶-(cis-hydroxyisopentenyl)adenosine, 2-methylthio-N6-(cis-hydroxyisopentenyl) adenosine, N⁶-glycinylcarbamoyladenosine, N⁶-threonylcarbamoyladenosine, 2-methylthio-N6-threonyl carbamoyladenosine, N⁶,N⁶-dimethyladenosine, 7-methyladenine, 2-methylthio-adenine, and 2-methoxy-adenine.

Modified nucleosides may include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N²,N²-dimethylguanosine, 8-oxo-guanosine, 7-methyl-8-oxo-guanosine, 1-methyl-6-thio-guanosine, N²-methyl-6-thio-guanosine, and N²,N²-dimethyl-6-thio-guanosine.

RNAs having an unmasked poly-A sequence may translated more efficiently than RNAs having a masked poly-A sequence. “Unmasked poly-A sequence” means that the poly-A sequence at the 3′ end of an RNA molecule ends with an A of the poly-A sequence and is not followed by nucleotides other than A located at the 3′ end, i.e. downstream, of the poly-A sequence. Furthermore, a long poly-A sequence of about 120 base pairs results in an optimal transcript stability and translation efficiency of RNA.

Therefore, in order to increase stability and/or expression of an RNA, the poly-A sequence may be modified, for example, for having a length of 10 to 500, for example 30 to 300, for example 65 to 200 and for example 100 to 150 adenosine residues. A poly-A sequence may have a length of approximately 120 adenosine residues. To further increase stability and/or expression of an RNA, the poly-A sequence can be unmasked.

Incorporation of a 3′-non-translated region (UTR) into the 3′-non-translated region of an RNA molecule can result in an enhancement in translation efficiency. A synergistic effect may be achieved by incorporating two or more of such 3′-non-translated regions. The 3′-non-translated regions may be autologous or heterologous to the RNA into which they are introduced. The 3′-non-translated region may be derived from the human β-globin gene.

A combination of the above-described modifications, i.e., incorporation of a poly-A sequence, unmasking of a poly-A sequence and incorporation of at least one 3′-non-translated region, may have a synergistic influence on the stability of RNA and increase in translation efficiency.

The expression of an RNA may be further increased by modification of the sequence encoding the peptide or protein, for example by increasing the GC-content to increase mRNA stability and/or by performing a codon optimization to enhance translation in cells.

### Nucleic Acid in LNPs

The frozen or freeze-dried LNPs obtained according to the methods disclosed herein contain a nucleic acid, such as a mRNA. The nucleic acid may encode a therapeutic agent. The nucleic acid, such as a mRNA, may be encapsulated with the LNPs.

Encapsulation rate of nucleic acid in LNPs may be measured by any method known in the art. For example, one may use a fluorescent probe as in the RiboGreen assay disclosed in the Examples section.

Encapsulation rate may be used to assess potential impact of a freezing or freeze-drying method on the stability or maintenance of the organizational structure of the LNPs.

The LNPs at the freezing step may contain a total amount of nucleic acid, such as mRNA, no lower than about 5%, or no lower than about 4%, or no lower than about 3%, or no lower than about 2%, or no lower than about 1% of the total amount of nucleic acid, such as mRNA, in LNPs in the liquid composition, as measured by the RiboGreen assay.

The LNPs at the freezing step may have a nucleic acid, such as mRNA, encapsulation rate no lower than 25%, or no lower than 22%, no lower than 20% of the nucleic acid encapsulation rate of the LNPs in the liquid composition.

The LNPs at the dried step may have a nucleic acid, such as mRNA, encapsulation rate, as measured by the RiboGreen assay, at 3-months no lower than 5%, or no lower than 2%, of the nucleic acid encapsulation rate of the LNPs at the dried step at TO, when stored at +5° C. A variation of the nucleic acid, such as mRNA, encapsulation rate in the LNPs after a 3-month storage at +5° C. of less than 5%, may be indicative of a low or reduced structural alteration of the LNPs during the storage period.

The LNPs at the dried step may contain a total amount of nucleic acid, such as mRNA, as measured by the RiboGreen assay, at 6-months no lower than about 5%, or no lower than about 2%, of the total amount of nucleic acid in the LNPs at the dried step at T0, when stored at +5° C. A variation of the total nucleic acid amount in the LNPs after a 6-month storage at +5° C. of less than 5% may be indicative of a low or reduced structural alteration of the LNPs during the storage period.

The LNPs at the dried step may have an nucleic acid, such as mRNA, encapsulation rate, as measured by the RiboGreen assay, at 6-months no lower than 10%, or no lower than 5%, of the nucleic acid encapsulation rate of at the dried step at TO, when stored at +5° C. A variation of the nucleic acid encapsulation rate in the LNPs after a 6-month storage at +5° C. of less than 10%, or no lower than 5%, may be indicative of a low or reduced structural alteration of the LNPs during the storage period.

The LNPs at the dried step may contain a total amount of nucleic acid, such as mRNA, as measured by the RiboGreen assay, at 11-months no lower than about 10%, or no lower than about 5%, of the total amount of nucleic acid in the LNPs at the dried step at TO, when stored at +5° C. A variation of the total nucleic acid amount in the LNPs after a 11-month storage at +5° C. of less than 10% may be indicative of a low or reduced structural alteration of the LNPs during the storage period.

The LNPs at the dried step may have an nucleic acid, such as mRNA encapsulation rate, as measured by the RiboGreen assay, at 11-months no lower than 10%, or no lower than 5%, of the nucleic acid encapsulation rate of at the dried step at TO, when stored at +5° C. A variation of the nucleic acid encapsulation rate in the LNPs after a 3-month storage at +5° C. of less than 10% or no lower than 5%, may be indicative of a low or reduced structural alteration of the LNPs during the storage period.

### Therapeutic Agent

A nucleic acid may be or encode a therapeutic agent. In some embodiments, a nucleic acid may be a mRNA encoding a therapeutic agent.

“Therapeutic agent” intends to refer to an active principle proposed to prevent or reduce the risk of occurrence of a disease condition or a symptom of a disease condition or to cure, or reduce the intensity of a disease condition, or to cure or reduce at least one symptom of a disease condition, in individual to whom it is administered. “Individual” intends to refer human and animals.

A therapeutic agent may be a peptide, a protein, a nucleic acid. In some embodiments, a therapeutic agent may be a nucleic acid. A nucleic acid may encode various therapeutic peptides or proteins.

A therapeutic agent may be a genome-editing polypeptide, a chemokine, a cytokine, a growth factor, an antibody, an enzyme, a structural protein, a blood protein, an hormone, a transcription factor, or an antigen.

In one embodiment, a therapeutic agent may be a genome-editing polypeptide. In some embodiments, the genome-editing polypeptide is a CRISPR protein, such as CRISPR/Cas9, a restriction nuclease, a meganuclease, a transcription activator-like effector protein (TALE, including a TALE nuclease, TALEN), or a zinc finger protein (ZF, including a ZF nuclease, ZFN). See, e.g., Int'l Pub. No. WO 2020/139783.

A therapeutic agent may be a cytokine or a chemokine suitable for stimulating or inhibiting an immune response, stimulating or preventing cell growth, or reducing an inflammation. Examples of suitable cytokine or chemokine include, but are not limited to, insulin, insulin-like growth factor, human growth hormone (hGH), tissue plasminogen activator (tPA), cytokines, such as interleukins (IL), e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, interferon (IFN) alpha, IFN beta, IFN gamma, IFN omega or IFN tau, tumor necrosis factor (TNF), such as TNF alpha and TNF beta, TNF gamma, TNF-related apoptosis-inducing ligand (TRAIL); lymphotoxin-β (LT-R), granulocyte colony-stimulating factor (G-CSF), granulocyte-macrophage colony-stimulating factor (GM-CSF), macrophage colony-stimulating factor (M-CSF), monocyte chemotactic protein-1 (MCP-1), and growth factors, such as vascular endothelial growth factor (VEGF). Also included is the production of erythropoietin or any other hormone growth factors.

In some embodiments, a therapeutic agent may be an antibody. As used herein, the term “antibody” refers to a whole antibody comprising two light chain polypeptides and two heavy chain polypeptides, or an antigen-binding fragment thereof. An antibody may be a monoclonal antibody (e.g., full length monoclonal antibody) that displays a single binding specificity and affinity for a particular epitope. An antigen-binding fragment may be a single chain antibody, a single chain Fv fragment (scFv), an Fd fragment, an Fab fragment, an Fab′ fragment, or an F(ab′)2 fragment. An antibody may recognize a tumor antigen or an infectious disease antigen, against which a protective or a therapeutic immune response is desired, e.g., antigens expressed by a tumor cell. Examples of antibodies include, for example, adalimumab, infliximab, rituximab, ipilimumab, tocilizumab, canakinumab, itolizumab, or tralokinumab.

In some embodiments, a therapeutic peptide or protein may be an enzyme with desirable uses for modulating metabolism or growth in a subject. In some embodiments, an enzyme may be administered to replace an endogenous enzyme that is absent or dysfunctional. In some embodiments, an enzyme may be used to treat a metabolic storage disease. A metabolic storage disease results from the systemic accumulation of metabolites due to the absence or dysfunction of an endogenous enzyme. Such metabolites include lipids, glycoproteins, and mucopolysaccharides. Examples of enzyme replacement therapy include lysosomal diseases, such as Gaucher disease, Fabry disease, MPS I, MPS II (Hunter syndrome), MPS VI and Glycogen storage disease type II.

A structural protein may be, for example, collagen, fibroin, fibrinogen, elastin, tubulin, actin, and myosin.

A blood protein may be, for example, thrombin, serum albumin, Factor VII, Factor VIII, insulin, Factor IX, Factor X, tissue plasminogen activator, protein C, von Wilebrand factor, antithrombin Ill, glucocerebrosidase, erythropoietin granulocyte colony stimulating factor (GCSF) or modified Factor VIII, anticoagulants, and the like.

An hormone may be for example insulin, thyroid hormone, gonadotrophine, trophic hormones, prolactin, oxytocin, dopamine, bovine somatotropin, leptins and the like.

Transcription factors (TFs) recognize specific DNA sequences to control chromatin and transcription, forming a complex system that guides expression of the genome. Several families of transcription factors exist, and members of each family may share structural characteristics. As example of transcription factors, one may cite helix-turn-helix (e.g., Oct-1), helix-loop-helix (e.g., E2A), zinc finger (e.g., glucocorticoid receptors, GATA proteins), basic protein-leucine zipper [cyclic AMP response element-binding factor (CREB), activator protein-1 (AP-1)], or β-sheet motifs [e.g. nuclear factor-κB (NF-κB)].

A therapeutic agent may be an antigen suitable for triggering an immune response, for example in cancer therapy or in a treatment of an infectious disease (e.g., a viral, bacterial, fungal, protozoal or parasitic infection.

According to some embodiments, compositions containing LNPs as disclosed herein which comprise antigens may therefore be immunogenic or vaccine compositions.

Antigen-containing compositions may vary in their valency. Valency refers to the number of antigenic components in the composition. The immunogenic or vaccine compositions may be monovalent or multivalent, i.e., divalent, trivalent compositions, or more. Multivalent compositions may comprise 2, 3, 4, 5, 6, 7, 8, 9, 10, 11, 12, 13, 14, 15, 16, 17, 18, 19, 20, or more antigens or antigenic moieties (e.g., antigenic peptides, etc.). The antigenic components may be on a single polynucleotide or on separate polynucleotides.

Compositions as disclosed herein may be used to protect, treat, or cure infection arising from contact with an infectious agent, such as bacteria, viruses, fungi, protozoa, and parasites.

Compositions as disclosed herein may be used to protect, treat, or cure cancer diseases.

According to some embodiments, a nucleic acid may encode for at least one antigen selected in the group consisting of bacterial antigens, viral antigens, and tumour antigens.

### Bacterial Antigens

The bacterium can be a Gram-positive bacterium or a Gram-negative bacterium. Bacterial antigens may be obtained from Acinetobacter baumannii, Bacillus anthracis, Bacillus subtilis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, coagulase Negative Staphylococcus, Corynebacterium diphtheria, Enterococcus faecalis, Enterococcus faecium, Escherichia coli, enterotoxigenic Escherichia coli (ETEC), enteropathogenic E. coli, E. coli 0157:H7, Enterobacter sp., Francisella tularensis, Haemophilus influenzae, Helicobacter pylori, Klebsiella pneumoniae, Legionella pneumophila, Leptospira interrogans, Listeria monocytogenes, Moraxella catarralis, Mycobacterium leprae, Mycobacterium tuberculosis, Mycoplasma pneumoniae, Neisseria gonorrhoeae, Neisseria meningitides, Proteus mirabilis, Proteus sps., Pseudomonas aeruginosa, Rickettsia rickettsii, Salmonella typhi, Salmonella typhimurium, Serratia marcesens, Shigella flexneri, Shigella sonnei, Staphylococcus aureus, Staphylococcus epidermidis, Staphylococcus saprophyticus, Streptococcus agalactiae, Streptococcus mutans, Streptococcus pneumoniae, Streptococcus pyogenes, Treponema pallidum, Vibrio cholerae, and Yersinia pestis.

### Viral Antigens

Viral antigens may be obtained from adenovirus; Herpes simplex, type 1; Herpes simplex, type 2; encephalitis virus, papillomavirus, Varicella-zoster virus; Epstein-barr virus; Human cytomegalovirus; Human herpesvirus, type 8; Human papillomavirus; BK virus; JC virus; Smallpox; polio virus, Hepatitis B virus; Human bocavirus; Parvovirus B19; Human astrovirus; Norwalk virus; coxsackievirus; hepatitis A virus; poliovirus; rhinovirus; Severe acute respiratory syndrome virus; Hepatitis C virus; yellow fever virus; dengue virus; West Nile virus; Rubella virus; Hepatitis E virus; Human immunodeficiency virus (HIV); Influenza virus, type A or B; Guanarito virus; Junin virus; Lassa virus; Machupo virus; Sabia virus; Crimean-Congo hemorrhagic fever virus; Ebola virus; Marburg virus; Measles virus; Mumps virus; Parainfluenza virus; Respiratory syncytial virus (RSV); Human metapneumovirus; Hendra virus; Nipah virus; Rabies virus; Hepatitis D; Rotavirus; Orbivirus; Coltivirus; Hantavirus, Middle East Respiratory Coronavirus; SARS-Cov-2 virus; Chikungunya virus; Zika virus; parainfluenza virus; Human Enterovirus; Hanta virus; Japanese encephalitis virus; Vesicular exanthernavirus; Eastern equine encephalitisor; or Banna virus.

In one embodiment, the antigen is from a strain of Influenza A or Influenza B virus or combinations thereof. The strain of Influenza A or Influenza B may be associated with birds, pigs, horses, dogs, humans or non-human primates.

The nucleic acid may encode a hemagglutinin protein or a fragment thereof. The hemagglutinin protein may be H1, H2, H3, H4, H5, H6, H7, H8, H9, H10, H11, H12, H13, H14, H15, H16, H17, H18, or a fragment thereof. The hemagglutinin protein may or may not comprise a head domain (HA1). Alternatively, the hemagglutinin protein may or may not comprise a cytoplasmic domain.

In embodiments, the hemagglutinin protein is a truncated hemagglutinin protein. The truncated hemagglutinin protein may comprise a portion of the transmembrane domain.

In some embodiments, the virus may be selected from the group consisting of H1N1, H3N2, H7N9, H5N1 and H10N8 virus or a B strain virus.

In another embodiment, the antigen may be from a Respiratory syncytial virus (RSV). Suitable RSV antigens may be from RSV A and/or RSV B strains. RSV antigens may be, for example, the fusion glycoprotein F protein, or the adhesion protein G protein.

In another embodiment, the antigen may be from a coronavirus such as SARS-Cov-1 virus, SARS-Cov-2 virus, or MERS-Cov virus. In some embodiments, an antigen may be a SARS-Cov2 antigen, such as a spike protein from SARS-Cov2.

### Tumour Antigens

An antigen may be a tumor antigen, i.e., a constituent of cancer cells such as a protein or a peptide expressed in a cancer cell. The term “tumor antigen” relates to proteins that are under normal conditions specifically expressed in a limited number of tissues and/or organs or in specific developmental stages and are expressed or aberrantly expressed in at least one tumor or cancer tissue. Tumor antigens include, for example, differentiation antigens, for example cell type specific differentiation antigens, i.e., proteins that are under normal conditions specifically expressed in a certain cell type at a certain differentiation stage and germ line specific antigens. For example, a tumor antigen is presented by a cancer cell in which it is expressed.

For example, tumor antigens may include the carcinoembryonal antigen, a 1-fetoprotein, isoferritin, and fetal sulphoglycoprotein, cc2-H-ferroprotein and γ-fetoprotein.

Other examples for tumor antigens that may be useful in the present disclosure are p53, ART-4, BAGE, beta-catenin/m, Bcr-abL CAMEL, CAP-1, CASP-8, CDC27/m, CD 4/m, CEA, the cell surface proteins of the claudin family, such as CLAUDIN-6, CLAUDIN-18.2 and CLAUDIN-12, c-MYC, CT, Cyp-B, DAM, ELF2M, ETV6-AML1, G250, GAGE, GnT-V, Gapl OO, HAGE, HER-2/neu, HPV-E7, HPV-E6, HAST-2, hTERT (or hTRT), LAGE, LDLR/FUT, MAGE-A, for example MAGE-A1, MAGE-A2, MAGE-A3, MAGE-A4, MAGE-A5, MAGE-A6, MAGE-A7, MAGE-A8, MAGE-A9, MAGE-A10, MAGE-A1 1, or MAGE-A12, MAGE-B, MAGE-C, MART-1/Melan-A, MC1 R, Myosin/m, MUC1, MUM-1, -2, -3, NA88-A, NF1, NY-ESO-1, NY-BR-1, pl 90 minor BCR-abL, Pm I/RARa, PRAME, proteinase 3, PSA, PSM, RAGE, RUI or RU2, SAGE, SART-1 or SART-3, SCGB3A2, SCP 1, SCP2, SCP3, SSX, SURVrVIN, TEL/AMLI, TPI/m, TRP-1, TRP-2, TRP-2/1 NT2, TPTE and WT, for example WT-1.

### Pharmaceutical Compositions and Uses Thereof

The freeze-dried or frozen LNPs obtained according to the methods disclosed herein may be used in a pharmaceutical composition. The pharmaceutical composition may comprise the freeze-dried or frozen LNPs as such or further formulated with at least one pharmaceutically acceptable excipient.

The present disclosure relates to freeze-dried or frozen LNPs obtained in accordance with a method as disclosed herein and comprising at least one nucleic acid, for use as a medicament.

The present disclosure relates to freeze-dried or frozen LNPs comprising at least a nucleic acid and, at least, as lipid components, a cationic ionizable lipid, a neutral lipid, and a steroid alcohol, or an ester thereof, optionally a PEG-lipid and comprising at least a nucleic acid, for use as a medicament, the freeze-dried LNPs being in freeze-dried micropellets or the frozen LNPs being in frozen micropellets.

A method for manufacturing a medicament, or a pharmaceutical composition, may comprise at least the steps of preparing freeze-dried LNPs in accordance with a method as disclosed herein, the LNPs comprising at least a nucleic acid.

A method for manufacturing a medicament, or a pharmaceutical composition, may comprise at least the steps of preparing frozen LNPs in accordance with a method as disclosed herein, the LNPs comprising at least a nucleic acid.

The method may further comprise a step of packaging the freeze-dried or frozen LNPs. The method may further comprise a step of formulating the freeze-dried or frozen LNPs with at least one pharmaceutically acceptable excipient. The method may further comprise a step of resuspending the freeze-dried LNPs in a pharmaceutically acceptable solvent or thawing the frozen LNPs.

An “pharmaceutically acceptable solvent” may be any solvent suitable for resuspending or dissolving the freeze-dried LNPs and pharmaceutically accepted for an enteral or parenteral administration to an individual in need thereof. A pharmaceutically acceptable solvent may be water for injection or a buffer, such as saline, a citrate, an histidine, or a phosphate buffer.

A pharmaceutical composition may be sterile.

General guidelines for the formulation and manufacture of pharmaceutical compositions and agents are available, for example, in Remington's The Science and Practice of Pharmacy, 21^{st} Edition, A. R. Gennaro; Lippincott, Williams & Wilkins, Baltimore, Md., 2006. Any pharmaceutically acceptable excipients may be used in a pharmaceutical composition, except insofar as an excipient may be incompatible with one or more components of an LNP.

Exemplary pharmaceutically acceptable excipients that may be used may be selected from diluents, such as water for injection, or physiological salt solutions, such as amino acids buffers (histidine, arginine, glycine, proline, glycylglycine), saline buffers (inorganic salts NaCl, calcium chloride), phosphate buffers, acetate buffers, citrate buffers, succinate buffers; sugars or polyalcohols such as dextrose, glycerol, ethanol, sucrose, trehalose, mannitol; surfactants such as Polysorbate 80, polysorbate 20, poloxamer 188; and the like, as well as combination thereof. In many cases, it will be preferable to include isotonic agents, such as sugars, polyalcohols, or sodium chloride in the composition, and formulation may also contain an anti-oxidant such as tryptamine and a stabilizing agent such as Tween 20 or 80, other solvents such as monohydric alcohols, such as ethanol, or isopropanol, and polyhydric alcohols such as glycols and edible oils such as soybean oil, coconut oil, olive oil, safflower oil, cottonseed oil, oily esters such as ethyl oleate, isopropyl myristate; binders, adjuvants, solubilizers, thickening agents, stabilizers, disintegrants, lubricating agents, buffering agents, emulsifiers, wetting agents, suspending agents, sweetening agents, colourants, flavours, preservatives, anti-oxidants, processing agents, drug delivery modifiers and enhancers such as calcium phosphate, magnesium stearate, talc, monosaccharides, disaccharides, starch, gelatin, cellulose, methylcellulose, sodium carboxymethyl cellulose, dextrose, hydroxypropyl-β-cyclodextrin, polyvinylpyrrolidone or polyethylene glycol. Pharmaceutically acceptable excipients may also include any and all solvents, dispersion media, coatings, anti-bacterial and anti-fungal agents, and the like that are physiologically compatible.

The frozen or freeze-dried LNPs may be administered by any suitable route, depending on parameters known in the art, such as the form of the composition (solid or liquid), the individual to be treated, the nature of the therapeutic agent contained in the LNPs, etc.

For example, a pharmaceutical composition with obtained frozen or freeze-dried LNPs may be administered systemically, orally, sublingually, intranasally, intradermally, or subcutaneously.

For parenteral administration in an aqueous solution, for example, the solution should be suitably buffered if necessary and the liquid diluent first rendered isotonic with sufficient saline or glucose. These aqueous solutions are especially suitable for intravenous, intramuscular, subcutaneous and intraperitoneal administration. In this connection, sterile aqueous media which can be employed will be known to those of skill in the art.

In some embodiments, a pharmaceutical composition with obtained frozen or freeze-dried LNPs may be suitable for subcutaneous administration.

A pharmaceutical composition comprising frozen or freeze-dried LNPs may be administrated through drug combination devices, such multi-chamber syringes, in which at least one chamber is containing the pharmaceutical composition in solid form and at least one chamber is containing a pharmaceutically acceptable solvent for suspending or dissolving the composition.

In some embodiments, the present disclosure relates to freeze-dried or frozen LNPs obtainable according to a method as disclosed herein and comprising at least a nucleic acid, for use as a medicament.

In some embodiments, the present disclosure relates to freeze-dried or frozen LNPs comprising at least, as lipid components, a cationic ionizable lipid, a neutral lipid, a steroid alcohol, or an ester thereof, and optionally a PEG-lipid, the frozen LNPs being in frozen micropellets or the freeze-dried LNPs being in freeze-dried micropellets, and comprising at least a nucleic acid, for use as a medicament.

In one of its objects, the present invention relates to frozen or freeze-dried LNPs as disclosed herein and comprising at least one nucleic acid encoding for an antigen from an Influenza A virus and/or an Influenza B virus for use in preventing or treating an Influenza A and/or an Influenza B virus infection.

In some embodiments, the present disclosure relates to frozen or freeze-dried LNPs comprising at least, as lipid components, a cationic ionizable lipid, a neutral lipid, a steroid alcohol, or an ester thereof, and optionally a PEG-lipid, the frozen LNPs being in frozen micropellets or the freeze-dried LNPs being in freeze-dried micropellets, and comprising at least one nucleic acid encoding for an antigen from an Influenza A virus and/or an Influenza B virus for use in preventing or treating an Influenza A and/or an Influenza B virus infection.

In one of its objects, the present invention relates to frozen or freeze-dried LNPs as disclosed herein and comprising at least one nucleic acid encoding for an antigen from a Respiratory syncytial A virus and/or a Respiratory syncytial B virus for use in preventing or treating a Respiratory syncytial A virus and/or a Respiratory syncytial B virus infection.

In some embodiments, the present disclosure relates to frozen or freeze-dried LNPs comprising at least, as lipid components, a cationic ionizable lipid, a neutral lipid, a steroid alcohol, or an ester thereof, and optionally a PEG-lipid, the frozen LNPs being in frozen micropellets or the freeze-dried LNPs being in freeze-dried micropellets, and comprising at least one nucleic acid encoding for an antigen from a Respiratory syncytial A virus and/or a Respiratory syncytial B virus for use in preventing or treating a Respiratory syncytial A virus and/or a Respiratory syncytial B virus infection.

In one of its objects, the present invention relates to frozen or freeze-dried LNPs as disclosed herein and comprising at least one nucleic acid encoding for an antigen from an Influenza A virus and/or an Influenza B virus for use as an immunogenic composition against an Influenza A virus and/or an Influenza B virus.

In some embodiments, the present disclosure relates to frozen or freeze-dried LNPs comprising at least, as lipid components, a cationic ionizable lipid, a neutral lipid, a steroid alcohol, or an ester thereof, and optionally a PEG-lipid, the frozen LNPs being in frozen micropellets or the freeze-dried LNPs being in freeze-dried micropellets, and comprising at least one nucleic acid encoding for an antigen from an Influenza A virus and/or an Influenza B virus for use as an immunogenic composition against an Influenza A virus and/or an Influenza B virus.

In one of its objects, the present invention relates to frozen or freeze-dried LNPs as disclosed herein and comprising at least one nucleic acid encoding for an antigen from a Respiratory syncytial A virus and/or a Respiratory syncytial B virus for use as an immunogenic composition against a Respiratory syncytial A virus and/or a Respiratory syncytial B virus.

In some embodiments, the present disclosure relates to frozen or freeze-dried LNPs comprising at least, as lipid components, a cationic ionizable lipid, a neutral lipid, a steroid alcohol, or an ester thereof, and optionally a PEG-lipid, the frozen LNPs being in frozen micropellets or the freeze-dried LNPs being in freeze-dried micropellets, and comprising at least one nucleic acid encoding for an antigen from a Respiratory syncytial A virus and/or a Respiratory syncytial B virus for use as an immunogenic composition against a Respiratory syncytial A virus and/or a Respiratory syncytial B virus.

In some embodiments, the present disclosure relates to frozen or freeze-dried LNPs obtainable according to a method as disclosed herein, and comprising at least one nucleic acid encoding for a SARS-Cov2 antigen for use in preventing or treating a SARS-Cov-2 infection.

In some embodiments, the present disclosure relates to frozen or freeze-dried LNPs comprising at least, as lipid components, a cationic ionizable lipid, a neutral lipid, a steroid alcohol, or an ester thereof, and optionally a PEG-lipid, the frozen LNPs being in frozen micropellets or the freeze-dried LNPs being in freeze-dried micropellets, and comprising at least one nucleic acid encoding for a SARS-Cov2 antigen for use in preventing or treating a SARS-Cov-2 infection.

In some embodiments, the present disclosure relates to frozen or freeze-dried LNPs obtainable according to a method as disclosed herein and comprising at least one nucleic acid encoding for a SARS-Cov2 antigen for use as an immunogenic composition against SARS-Cov-2.

In some embodiments, the present disclosure relates to frozen or freeze-dried LNPs comprising at least, as lipid components, a cationic ionizable lipid, a neutral lipid, a steroid alcohol, or an ester thereof, and optionally a PEG-lipid, the frozen LNPs being in frozen micropellets or the freeze-dried LNPs being in freeze-dried micropellets, and comprising at least one nucleic acid encoding for a SARS-Cov2 antigen for use as an immunogenic composition against SARS-Cov-2.

In some embodiments, the present disclosure relates to a use of frozen or freeze-dried LNPs obtainable according to a method as disclosed herein and comprising at least one nucleic acid in the manufacture of a medicament.

In some embodiments, the present disclosure relates to a use of frozen or freeze-dried LNPs comprising at least, as lipid components, a cationic ionizable lipid, a neutral lipid, a steroid alcohol, or an ester thereof, and optionally a PEG-lipid, the frozen LNPs being in frozen micropellets or the freeze-dried LNPs being in freeze-dried micropellets, and comprising at least one nucleic acid in the manufacture of a medicament.

In some embodiments, the present disclosure relates to a method for preventing and/or treating a disorder in an individual in need thereof, the method comprising at least the steps of:
resuspending freeze-dried LNPs obtainable according to a method as disclosed herein in a pharmaceutically acceptable solvent or thawing the frozen the LNPs, the frozen or freeze-dried LNPs comprising at least one nucleic acid presumed to be active against said disorder, to obtain resuspended or thawed LNPs and
administering to said individual the resuspended or thawed LNPs.

In some embodiments, the present disclosure relates to a method for preventing and/or treating a disorder in an individual in need thereof, the method comprising at least the steps of:
resuspending freeze-dried LNPs in a pharmaceutically acceptable solvent or thawing frozen LNPs, the LNPs comprising at least, as lipid components, a cationic ionizable lipid, a neutral lipid, a steroid alcohol, or an ester thereof, and optionally a PEG-lipid, the frozen LNPs being in frozen micropellets or the freeze-dried LNPs being in freeze-dried micropellets, the frozen or freeze-dried LNPs comprising at least one nucleic acid presumed to be active against said disorder, to obtain resuspended or thawed LNPs and
administering to said individual the resuspended or thawed LNPs.

In some embodiments, a nucleic acid may an RNA. In some embodiments, a RNA may be an mRNA.

Also is disclosed a method for delivering LNPs to an individual, comprising steps of: (i) suspending or dissolving freeze-dried LNPs in a pharmaceutically acceptable solvent to obtain a solution of LNPs or thawing a frozen LNPs, and (ii) administering the solution of LNPs in an individual in need thereof. Step (ii) ideally takes place within 24 hours of step (i) e.g., within 12 hours, within 6 hours, within 3 hours, or within 1 hour after reconstitution of the LNPs in a liquid formulation.

It is to be understood that the disclosure encompasses all variations, combinations, and permutations in which at least one limitation, element, clause, descriptive term, etc., from at least one of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group. It should be understood that, in general, where the disclosure, or aspects of the disclosure, is/are referred to as comprising particular elements, features, etc., they also encompass embodiments consisting, or consisting essentially of, such elements, features, etc. For purposes of simplicity those embodiments have not in every case been specifically set forth in so many words herein. It should also be understood that any embodiment or aspect of the disclosure can be explicitly excluded from the claims, regardless of whether the specific exclusion is recited in the specification. The publications and other reference materials referenced herein to describe the background of the disclosure and to provide additional detail regarding its practice are hereby incorporated by reference.

The following examples are provided for purpose of illustration and not limitation.

### EXAMPLES

### Example 1: Material and Methods

### Preparation of mRNA-Containing Lipid Nanoparticles (LNPs)

LNPs were prepared with DLin-MC3-DMA (or (6Z,9Z,28Z,31Z)-heptatriacont-6,9,28,31-tetraene-19-yl 4-(dimethylamino)butanoate from SAI Life Science as ionizable cationic lipid; DSPC as neutral lipid (1,2-distearoyl-sn-glycero-3-phosphocholine—Avanti-Polar Lipids: ref 850365), cholesterol as steroid alcohol—Avanti-Polar Lipids ref 700000P, and DMG-PEG 2000 1,2-dimyristoyl-sn-glycero-3-phosphoethanolamine-N-[methoxy(polyethylene glycol)-2000] Avanti Polar Lipids ref 880150P as PEG-lipid.

Lipid components of the LNPs were dissolved into ethanol. The molar ratio of neutral lipid:steroid alcohol:PEG-lipid:ionizable cationic lipid was 10:38.5:1.5:50.

The encapsulated mRNA was a non-replicative mRNA luciferase (mRNA-Luc; Ref.: L-7602 TriLink™ Biotechnologies. mRNA was prepared in a citrate buffer (pH 4.0) at a concentration of 900 μg/mL of mRNA.

mRNA and lipids were mixed at a ratio of 3:1. The charge ratio used in all experiments was 6:1.

The LNPs were prepared according to the method described thereafter.

A stock solution of LNPs at 100 μg/mL mRNA and 3 mg/mL of total lipids was obtained, after purification and formulation.

### Preparation of the Organic Phase

### Preparation of 6 mL of Organic Phase for LNPs Formulation

46 mg of DSPC, 24 mg of DMG-PEG 2000 and 88 mg of cholesterol were dissolved with 3971 μL of ethanol. Then 1872 μL of DLin-MC3-DMA stock solution (100 mg/mL in ethanol) was added to obtain 20 mg/mL of lipid phase solution.

### Preparation of the Aqueous Phase

Preparation of 1.8 mL of aqueous phase for LNPs

mRNA concentration to be used in aqueous phase was calculated to obtain a Lipid Nitrogen/mRNA Phosphate charge ratio of 6/1 (N/P=6/1). This concentration was determined from the ionizable cationic lipid concentration assuming 1 μg mRNA corresponds to 0.003 μmol of phosphate. Since 1.5 mL of aqueous solution is needed to make 2 mL of LNPs when using a ratio of aqueous solution to ethanol solution of 3:1 with the NanoAssemblR® (Nanoassemblr Benchtop from Precision Nanosystem; Belliveau et al., Molecular Therapy—Nucleic Acids (2012)), the required mRNA concentration was calculated to be 900 μg/mL.

The mRNA solution was prepared in a 50 mM citrate buffer pH 4.0.

### LNPs Preparation

LNPs were prepared using a NanoAssemblR equipment according to manufacturer recommendations.

The aqueous and organic phases were each loaded in a syringe suitable for NanoAssemblR according to manufacturer recommendations. The flow rate was set up at a ratio: 3:1 and total flow rate: 4 ml/min. The aqueous and lipid phases were then mixed to obtain the LNPs.

### LNPs Purification and Harvest

The obtained LNPs were dialyzed against a citrate buffer (50 mM-pH 4.0) to remove residual ethanol.

### Preparation of the LNPs-Containing Formulation for Freezing and Freeze-Drying

LNPs-formulations for freezing and freeze-drying were prepared by adding the excipients to the LNPs prepared as above disclosed, prior to storage of final products.

First a dialysis step was carried to remove the ethanol from encapsulation process with citrate buffer. Then, a second dialysis was performed with a Tris-buffer (50 mM), pH 7.5. Then, trehalose as cryoprotectant was added (500 mM). The formulated LNPs were then sterile-filtered (0.22 μm) before being filled in vials, frozen or freeze-dried.

### Freezing Process

The formulated LNPs containing mRNA were submitted to two types of freezing processes: freezing in vials or freezing by spray-freezing.

Freezing in vials was carried by filling 0.5 mL of formulated LNPs (obtained as above indicated) in 3-mL type 1 glass vials with lyo-stoppers (West ref 7002-4333). Vials were frozen at minus 80° C. or minus −20° C. for liquid process. Vials were frozen on freeze-dryer shelves regulated at −45° C. under atmospheric pressure. Frozen vials were stored at, respectively, minus 80° C. or minus 20° C. until use.

Spray-freezing was carried out by prilling (electro-magnetic droplet-stream nozzle—Meridion Technologies GmbH, Mullheim, Germany+prilling tower Gatt, Binzen, Germany) a liquid jet of formulated LNPs in a jacketed chamber (prilling tower) cooled by direct nebulization/vaporization of liquid nitrogen (Adali et al., Processes 2020, 8, 709; Wanning et al., Int J Pharm. 2015; 488(1-2):136-153; WO 2013/050156 A1; WO 2013/050159 A1; or WO 2016/012414 A1). The atmosphere in the cooled chamber was cooled below minus 105° C., the liquid flowrate was 20 mL/min, the vibration frequency was 4000 Hz, and nozzle diameter 300 μm. The height of the tower was 160 cm. Frozen micropellets (or microbeads) were poured on −50° C. pre-cooled trays and freeze dried at 50 μBar (freeze dryer SMH90, Elancourt, France). The frozen micropellets were harvested and stored at minus 80° C. until use.

### Freeze-Drying (Lyophilization) Process

The LNPs containing mRNA were submitted to two types of freeze-drying processes: freeze-drying in vials (or conventional lyophilization) and spray-freeze drying (or prilling: freezing of droplets and then drying).

### Freeze-Drying in Vials (Conventional Lyophilization)

Freeze-drying in vial was carried by filling 0.5 mL of formulated LNPs (obtained as above indicated) in 3-mL type 1 glass vials with lyo-stoppers (West ref 7002-4333). Vials were then lyophilized in Scientific Lyostar freeze dryer as follows:

| Steps | | Temperature | Pressure | Duration (hrs) |
|---|---|---|---|---|
| loading | | 20° C. | Atm | NA |
| freezing | decrease | +20° C. → −45° C. | Atm | 01 h 00 |
| | landing | −45° C. | Atm | 01 h 30 |
| | venting | −45° C. | 50 μbars | 00 h 45 |
| Primary | heating | −45° C. → −30° C. | 50 μbars | 02 h 00 |
| desiccation | landing | −30° C. | 50 μbars | 23 h 00 |
| | heat | −30° C. → 0° C. | 50 μbars | 03 h 00 |
| Secondary | landing 1 | 0° C. | 50 μbars | 05 h 00 |
| desiccation | | | | |

Stoppering at 800 mbar under nitrogen
NA: Not Applicable

Atm: Atmospheric pressure

Freeze-dried cakes of formulated LNPs were obtained and stored at +5° C., for 0, 1, 2, 3, 6 or 11-months before analysis.

### Spray Freeze-Drying Process

Spray-freezing was carried out by prilling (electromagnetic droplet-stream generator) as above indicated.

The frozen micropellets were harvested and then dried on freeze-dryer shelves. Frozen micropellets were poured on −50° C. pre-cooled trays and freeze dried at 50 μBar (freeze dryer USIFROID SMH90, Elancourt, France).

The freeze-dried micropellets were harvested, filled in 5-mL type 1 glass vials with lyo-stoppers (West ref 7002-4333) (100 mg/vial) with a powder Quantos dosage system—Mettler Toledo, Columbus, Ohio, US) and stored at +5° C. until use. Freeze-dried LNPs were stored for 0, 1, 2, 3, 6 or 11-months before analysis.

### Resuspension of the LNPs

Frozen LNPs were thawed at room temperature.

The freeze-dried LNPs were resuspended in water for injection to obtain a resuspended LNP solution at 100 μg/mL RNA before the analytical measurements or in vivo assays as described thereafter.

### Analytical Methods

The following analytical methods were used to characterize the LNPs after the freezing and the freeze-drying processes.

### LNP Size and Concentration

LNPs' sizes and concentration were measured by NTA (Nanoparticles Tracking Analysis).

NTA measures were carried out with NanoSight NS300 (Malvern) and Nano Sample Assistant (Malvern) equipment according to manufacturer recommendations. Frozen LNPs were thawed at room temperature and freeze-dried LNPs were resuspended in water (0.5 mL) as above indicated. Resuspended or thawed LNPs were further diluted (1/2000) in a Tris buffer (Tris 50 mM) and distributed in 96-wells plates with a dilution robot Janus from Perkin Elmer. LNPs' size (mean size and mode size) and concentration were then measured (camera level 15—detect threshold 5). SURF-CAL™ Particle Size Standard (Thermo Scientific PD-047B—Size 47±2 nm; concentration 1×10¹⁰ particles/mL) was used as control (camera level 15—detect threshold 3). NTA Results presented here are the average results of samples in triplicate measurement.

LNPs obtained after 0.22 μm filtration and before freezing or freeze-drying were used as control.

### RNA Encapsulation Rate

The percentage of encapsulated mRNA and concentration of mRNA in LNPs were measured using the Quant-iT Ribogreen RNA reagent kit according to manufacturer recommendations (Invitrogen Detection Technologies) and quantified with a fluorescent microplate reader. Standard curves using RiboGreen RNA standard (100 μg/ml) and internal control (Clean Cap Fluo mRNA—ref L7602—1 mg/mL) with and without Triton X100 (0.5%) were prepared in a TE buffer (Tris 10 mM, EDTA 1 mM, pH 7.5), respectively from 1 to 0.03125 μg/mL of RNA for the standard curve and from 100 to 3.125 μg/mL mRNA for internal control)

Frozen LNPs were thawed at room temperature and freeze-dried LNPs were resuspended in water (0.5 mL) as above indicated.

For quantification of non-encapsulated RNA, LNPs were serially diluted in Tris/EDTA assay buffer (10 mM Tris-HCl, 1 mM EDTA, pH 7.5). For quantification of total amount of RNA, LNPs were serially diluted in Tris/EDTA assay buffer (10 mM Tris-HCl, 1 mM EDTA, pH 7.5) containing 0.5% (v/v) Triton X100.

Controls and samples were distributed in 96-wells plates.

Ribogreen dye (200× diluted) was added to the samples (50/50 mix; sample/Ribogreen reagent), mixed thoroughly and incubated 5 min at room temperature in the dark. Fluorescence was measured on a SpectraMax plate reader (excitation and emission wavelengths: 485 and 528 nm).

LNPs obtained after 0.22 μm filtration and before freezing or freeze-drying were used as control. After filtration, LNPs were sampled to be used as LNPs control not submitted to any process (“No freezing” and “No freeze-drying”).

mRNA Integrity

Capillary Electrophoresis was used to determine mRNA integrity in LNPs using Bioanalyzer 2100 (AGILENT TECHNOLOGIES) according to manufacturer recommendations (“Quick Start Guide RNA 6000 Pico Kit G2938-90049” Rev. C Edition August/2013).

Decapsulation of mRNA was carried by mixing samples with a TE buffer −0.5% Triton X100 in a sample:buffer volume ratio of 1:9. Samples were further diluted (1:40 final) and thermally denatured (70° C.) for 2 minutes.

mRNA samples were then loaded in the gel according to manufacturer recommendations (“Quick Start Guide RNA 6000 Pico Kit G2938-90049” Rev. C Edition August/2013).

LNPs obtained after 0.22 μm filtration and before freezing or freeze-drying were used as control.

### In Vivo Bioluminescence

LNPs containing mRNA-luc were prepared as described in Example 1. LNPs were freeze-dried either by conventional lyophilization in vials or by spray-freeze drying to obtain spray-freeze dried micropellets (see Example 1). Freeze-dried (or lyophilized) LNPs were stored at +5° C. for 320 days before being resuspended in water for injection and injected by intramuscular route to mice (5 animals per condition—SKH1 hairless female mice, 6-weeks old). Injections were done in the right quadriceps muscle. Each mouse received 3 μg of mRNA (35 μL injected). After LNPs mRNA-Luc injections, measures were acquired at different time points: T0h, T6h, and T24h. Control mice received LNPs without mRNA.

After intramuscular injection, the mice were imaged with IVIS Spectrum CT device to get background bioluminescence signal (T0). To generate bioluminescence, the mice were administered with 150 mg/kg of D-luciferin by intraperitoneal route at T6h bioluminescence acquisitions and at D1 for the T24h acquisition, and D3 at T72h. Fifteen minutes later, the mice will be anesthetized (isoflurane) and the acquisitions of bioluminescence signal (luciferase) were performed. Acquisitions were performed in the right quadriceps region.

To quantify bioluminescence signal, Regions of Interest (ROI) were defined for each animal. The size and position of the ROIs were adjusted to the quadriceps muscle. For each animal the same size of ROI was used.

Quantification was performed thanks to ROIs measurements tool from Living Image software. Results were expressed as Total flux (photons/second).

### Example 2: Effect of Freezing Process on LNPs Stability

### Design of Experiment

LNPs containing mRNA-Luc were prepared as described in Example 1.

In a first set of experiments LNPs were frozen in vials at −20° C. and −80° C. or spray-frozen in micropellets at 80° C. (see Example 1).

Frozen LNPs were thawed before acquisition of measures.

Size and concentration of LNPs were determined by NTA (see Example 1).

RNA encapsulation rate was determined by Quant-iT™ RiboGreen® RNA Assay (see Example 1).

### Results

LNPs mean and mode diameters, and concentrations obtained by NTA are summarized in the Table 1:

TABLE 1
LNPs mean and mode diameters, and
concentrations obtained by NTA

| | | Mean | Mode | | Concentration |
|---|---|---|---|---|---|
| Temperature | Freezing | diameter - | diameter - | Std. | (Log |
| (° C.) | process | nm | nm | Dev. | particles/ml) |
| No freezing | NA | 117 | 93 | 38 | 12.7 |
| −20° C. | vial | 152 | 135 | 53 | 12.1 |
| −80° C. | vial | 136 | 118 | 47 | 12.3 |
| −80° C. | spray- | 131 | 99 | 46 | 12.3 |
| | freezing | | | | |

NA: not applicable
Std. Dev.: standard deviation.

Total RNA and RNA encapsulation rates are summarized in the Tables 2 and 3: PGP-30

TABLE 2
LNPs total RNA content

| Temperature | Freezing | Total RNA | |
|---|---|---|---|
| (° C.) | process | (μg/ml) | Std. Dev. |
| No freezing | NA | 98 | 3 |
| −20° C. | vial | 95 | 6 |
| −80° C. | vial | 102 | 12 |
| −80° C. | spray-freezing | 98 | 10 |

NA: not applicable
Std. Dev.: standard deviation.

TABLE 3
LNPs RNA encapsulation rate

| | | RNA |
|---|---|---|
| Temperature | Freezing | encapsulation |
| (° C.) | process | rate (%) |
| No freezing | NA | 74 |
| −20° C. | vial | 46 |
| −80° C. | vial | 55 |
| −80° C. | spray-freezing | 58 |

NA: not applicable

The data shows that spray-frozen LNPs at −80° C. tend to have an increased mRNA encapsulation rate compared to LNPs frozen in vials at −80° C. or at −20° C.

This data shows that spray-freezing of LNPs allow to improve stability of frozen LNPs and mRNA encapsulation rate.

Taken together, the results show that spray-freezing of LNPs allow to improve stability of frozen LNPs, by reducing LNPs aggregation, and maintaining mRNA encapsulation rate.

Reduction of LNPs aggregation can be beneficial at the time of injection as aggregates constituting large lumps may cause adverse reactions such as pain. Further, the maintenance of a good mRNA encapsulate rate will improve the corresponding protein expression and therefore the therapeutic sought effect.

### Example 3: Effect of Freeze-Drying Process on LNPs Stability

### Design of Experiment

LNPs containing mRNA-luc were prepared as described in Example 1.

LNPs were conventionally freeze-dried in vials (Conventional) or spray-freeze dried by prilling (SFD) (see Example 1). Lyophilizates were stored at +5° C. for TO, 3, 6 or 11-months.

Lyophilized LNPs were resuspended in water for injection before acquisition of measures.

Size and concentration of resuspended LNPs were determined by NTA (see Example 1).

RNA encapsulation rate of resuspended LNPs was determined by Quant-iT™ RiboGreen® RNA Assay (see Example 1).

### Results

Resuspended LNPs mean and mode diameters, and concentrations obtained by NTA are summarized in the Tables 4, 5 and 6:

TABLE 4
LNPs mean diameters obtained by NTA after storage of lyophilized LNPs at +5° C.

| | Time | | | | | | | |
|---|---|---|---|---|---|---|---|---|
| | 0 | | 3 months | | 6 months | | 11 months | |
| | Mean | | Mean | | Mean | | Mean | |
| | diameter | Std. | diameter | Std. | diameter | Std. | diameter | Std. |
| Process | (nm) | Dev. | (nm) | Dev. | (nm) | Dev. | (nm) | Dev. |
| No freeze- | 117 | 38 | 120 | 41 | 124 | 41 | NA | NA |
| drying | | | | | | | | |
| Conventional | 135 | 48 | 132 | 46 | 135 | 47 | 137 | 52 |
| SFD | 136 | 47 | 131 | 47 | 134 | 46 | 129 | 48 |

NA: not available
Std. Dev.: standard deviation.

TABLE 5
LNPs mode diameters obtained by NTA
after storage of lyophilized LNPs at +5° C.

| | Time | | | |
|---|---|---|---|---|
| | 0 | 3 months | 6 months | 11 months |
| Process | Mode diameter (nm) | | | |
| No freeze- | 96 | 94 | 97 | NA |
| drying | | | | |
| Conventional | 104 | 100 | 102 | 111 |
| SFD | 104 | 94 | 106 | 97 |

NA: not available
Std. Dev.: standard deviation.

TABLE 6
LNPs concentration (Log particles/mL)
obtained by NTA after storage of lyophilized LNPs at +5° C.

| | Time | | | |
|---|---|---|---|---|
| | 0 | 3 months | 6 months | 11 months |
| Process | Concentration | | | |
| No freeze-drying | 12.3 | 12.2 | 12.2 | NA |
| Conventional | 12.2 | 12.0 | 12.0 | 12.1 |
| SFD | 12.2 | 12.1 | 12.1 | 12.2 |

NA: not available
Concentration: concentration expressed as Log particles/mL

Taken together, the above-data tends to show that size of the spray-freeze dried and conventionally lyophilized LNPs are stable over time, in particular when stored at +5° C.

Resuspended LNPs RNA encapsulation rates and total RNA are summarized in the Tables 7 and 8:

TABLE 7
LNPs total RNA determined after storage
of lyophilized LNPs at +5° C.

| | Time | | | |
|---|---|---|---|---|
| | 0 | 3 months | 6 months | 11 months |
| Process | Total RNA | | | |
| No freeze-drying | 95 | 98 | NA | NA |
| Conventional | 90 | 100 | 85 | 81 |
| SFD | 84 | 97 | 92 | 85 |

NA: not available

TABLE 8
LNPs RNA encapsulation rates determined
after storage of lyophilized LNPs at +5° C.

| | Time | | | |
|---|---|---|---|---|
| | 0 | 3 months | 6 months | 11 months |
| Process | Encaps. Rate (%) | | | |
| No freeze-drying | 75 | 71 | NA | NA |
| Conventional | 50 | 46 | 45 | 40 |
| SFD | 48 | 48 | 49 | 46 |

Encaps. Rate (%): mRNA encapsulation rate
NA: not available

The data shows that spray-freeze dried LNPs tend to have an mRNA encapsulation rate stabler than the mRNA encapsulation rate of LNPs conventionally lyophilized, in particular after 11-months of storage, when compared with the mRNA encapsulation rate in the liquid, pre-freeze-drying stage.

This data shows that spray-freeze drying of LNPs allow to improve stability of mRNA encapsulation rate.

Taken together, the results show that spray-freeze drying of LNPs allow to maintain stability of LNPs, by preventing or reducing LNPs aggregation, and by maintaining mRNA encapsulation rate.

Reduction of LNPs aggregation can be beneficial at the time of injection as aggregates constituting large lumps which may cause adverse reactions such as pain. Further, the maintenance of a good mRNA encapsulate rate will improve the corresponding protein expression and therefore the therapeutic sought effect.

### Example 4: Effect of Freezing and Freeze-Drying Process on mRNA Integrity

### Design of Experiment

LNPs containing mRNA-luc were prepared as described in Example 1.

LNPs were conventionally freeze-dried in vials (Conventional lyophilization) or spray-freeze dried by prilling (SFD) (see Example 1). Lyophilizates were stored at +5° C. for T0, 3, or 6-months.

mRNA integrity was measured as indicated in Example 1.

### Results

The expected size of the luciferase mRNA (mRNA-Luc; Ref.: L-7602 TriLink™ Biotechnologies) is 1941 bases.

Whatever the lyophilizing process, conventional lyophilization vs spray-freeze drying, or the duration of the storage at +5° C., 0, 3 or 6-months, the integrity of the mRNA was maintained.

### Example 5: Effect of Freeze-Drying Process on In Vivo mRNA Expression

### Design of Experiment

LNPs containing mRNA-Luc were prepared as described in Example 1.

LNPs were conventionally freeze-dried in vials (Conventional lyophilization) or spray-freeze dried by prilling (SFD) (see Example 1). Lyophilizates were stored at +5° C. for 320 days until further use.

3 groups of mice were treated: (1) LNPs with mRNA and conventionally lyophilized, (3) LNPs with mRNA and spray-freeze dried, and (3) LNPs without mRNA and without freeze drying before use.

The bioluminescence was measured as indicated in Example 1.

### Results

The obtained bioluminescence results are summarized in the Table 9:

TABLE 9
Bioluminescence according to LNPs lyophilization process

| | Conventional Lyo. | | SFD | |
|---|---|---|---|---|
| | Total Flux | | Total Flux | |
| Time | (p/s). | Std. Dev. | (p/s). | Std. Dev. |
| 6 hours | 1.33E07 | 2.94E5 | 5.30E8 | 1.30E7 |
| 24 hours | 5.62E5 | 1.56E4 | 3.19E7 | 8.41E5 |
| 72 hours | 1.37E6 | 3.70E4 | 2.16E7 | 5.23E5 |

Conventional Lyo.: conventional lyophilization
SFD: spray-freeze drying
Std. Dev.: standard deviation.

As shown by the above-data, at 6- and 24-hours post-injection, the bioluminescence, and therefore the level of expression of the luciferase, is significantly greater in mice injected with spray-freeze dried LNPs vs conventionally lyophilized LNPs.

At 6 hours, the level of bioluminescence obtained with the spray freeze-dried LNPs was about 40 times the level of bioluminescence obtained with conventionally lyophilized LNPs. Three days after the injection, the level of bioluminescence obtained with the spray freeze-dried LNPs was still about 15-times the level of bioluminescence obtained with the conventionally lyophilized LNPs.

An enhanced expression of protein encoded by the mRNA will find beneficial interest for therapeutic application, as for example in immunization and vaccine application where an enhanced expression of an antigen may assist in obtaining an enhanced immune response.

### REFERENCES

Adali M B, Barresi A A, Boccardo G, Pisano R. Spray Freeze-Drying as a Solution to Continuous Manufacturing of Pharmaceutical Products in Bulk. Processes. 2020; 8(6):709. https://doi.org/10.3390/pr8060709
Ali M E, Lamprecht A. Spray freeze drying as an alternative technique for lyophilization of polymeric and lipid-based nanoparticles. Int J Pharm. 2017; 516(1-2):170-177. doi:10.1016/j.ijpharm.2016.11.023
Ball, R. L., Bajaj, P., & Whitehead, K. A. (2016). Achieving long-term stability of lipid nanoparticles: examining the effect of pH, temperature, and lyophilization. Int J Nanomedicine, 12, 305-315. https://doi.org/10.2147/IJN.S123062
Belliveau N M, Huft J, Lin P J, et al. Microfluidic Synthesis of Highly Potent Limit-size Lipid Nanoparticles for In Vivo Delivery of siRNA. Mol Ther Nucleic Acids. 2012; 1(8):e37. Published 2012 Aug. 14. doi:10.1038/mtna.2012.28
Brandenberger H, Widmer F (1998) A new multinozzle encapsulation immobilisation system to produce uniform beads of alginate. J Biotechnol 63:73-80.
Brunelle J L, Green R. In vitro transcription from plasmid or PCR-amplified DNA. Methods Enzymol. 2013; 530:101-114. doi:10.1016/B978-0-12-420037-1.00005-1
Crommelin D J A, Anchordoquy T J, Volkin D B, Jiskoot W, Mastrobattista E. Addressing the Cold Reality of mRNA Vaccine Stability. J Pharm Sci. 2021; 110(3):997-1001. doi:10.1016/j.xphs.2020.12.006
Fukushige K, Tagami T, Naito M, et al. Developing spray-freeze-dried particles containing a hyaluronic acid-coated liposome-protamine-DNA complex for pulmonary inhalation. Int J Pharm. 2020; 583:119338. doi:10.1016/j.ijpharm.2020.119338
Geall A J, Mandl C W, Ulmer J B. RNA: the new revolution in nucleic acid vaccines. Semin Immunol. 2013; 25(2):152-159. doi:10.1016/j.smim.2013.05.001;
Khurana, A., Allawadhi, P., Khurana, I., Allwadhi, S., Weiskirchen, R., Banothu, A. K., Chhabra, D., Joshi, K., & Bharani, K. K. (2021). Role of nanotechnology behind the success of mRNA vaccines for COVID-19. Nano today, 38, 101142. https://doi.org/10.1016/j.nantod.2021.101142
Maier M A, Jayaraman M, Matsuda S, Liu J, Barros S, Querbes W, Tam Y K, Ansell S M, Kumar V, Qin J, Zhang X, Wang Q, Panesar S, Hutabarat R, Carioto M, Hettinger J, Kandasamy P, Butler D, Rajeev K G, Pang B, Charisse K, Fitzgerald K, Mui B L, Du X, Cullis P, Madden T D, Hope M J, Manoharan M, Akinc A. Biodegradable lipids enabling rapidly eliminated lipid nanoparticles for systemic delivery of RNAi therapeutics. Mol Ther. 2013 August; 21(8):1570-8. doi: 10.1038/mt.2013.124. Epub 2013 Jun. 25. PMID: 23799535; PMCID: PMC3734658.
Meesters, G. M. H. (1992) Mechanisms of droplet formation. Thesis, Technische Universiteit Delft, Delft University Press.
Rayleigh, L. (1878), On The Instability Of Jets. Proceedings of the London Mathematical Society, s1-10: 4-13. https://doi.org/10.1112/plms/s1-10.1.4
Reichmuth, A. M., Oberli, M. A., Jaklenec, A., Langer, R., & Blankschtein, D. (2016). mRNA vaccine delivery using lipid nanoparticles. Therapeutic delivery, 7(5), 319-334. https://doi.org/10.4155/tde-2016-0006
Thi, T., Suys, E., Lee, J. S., Nguyen, D. H., Park, K. D., & Truong, N. P. (2021). Lipid-Based Nanoparticles in the Clinic and Clinical Trials: From Cancer Nanomedicine to COVID-19 Vaccines. Vaccines, 9(4), 359. https://doi.org/10.3390/vaccines9040359
Trenkenschuh E, Friess W. Freeze-drying of nanoparticles: How to overcome colloidal instability by formulation and process optimization. Eur J Pharm Biopharm. 2021 August; 165:345-360. doi: 10.1016/j.ejpb.2021.05.024. Epub 2021 May 27. PMID: 34052428.
Schoenmaker, L., Witzigmann, D., Kulkarni, J. A., Verbeke, R., Kersten, G., Jiskoot, W., & Crommelin, D. (2021). mRNA-lipid nanoparticle COVID-19 vaccines: Structure and stability. Int J Pharm, 601, 120586. https://doi.org/10.1016/j.ijpharm.2021.120586
U.S. Pat. No. 9,512,073—Amino acid-, peptide- and polypeptide-lipids, isomers, compositions, and uses thereof
U.S. Pat. No. 10,201,618—Alkenyl substituted 2,5-piperazinediones, compositions, and uses thereof
U.S. Pat. No. 4,373,071—Solid-phase synthesis of polynucleotides
U.S. Pat. No. 4,401,796—Solid-phase synthesis of polynucleotides
U.S. Pat. No. 4,415,732—Phosphoramidite compounds and processes
U.S. Pat. No. 4,458,066—Process for preparing polynucleotides
U.S. Pat. No. 4,500,707—Nucleosides useful in the preparation of polynucleotides
U.S. Pat. No. 4,668,777—Phosphoramidite nucleoside compounds
U.S. Pat. No. 4,973,679—Process for oligonucleotide synthesis using phosphoramidite intermediates
U.S. Pat. No. 5,047,524—Automated system for polynucleotide synthesis and purification
U.S. Pat. No. 5,132,418—Process for preparing polynucleotides
U.S. Pat. No. 5,153,319—Process for preparing polynucleotides
U.S. Pat. No. 5,262,530—Automated system for polynucleotide synthesis and purification
U.S. Pat. No. 5,700,642—Oligonucleotide sizing using immobilized cleavable primers
Wanning S, Süverkrüp R, Lamprecht A. Pharmaceutical spray freeze drying. Int J Pharm. 2015; 488(1-2):136-153. doi:10.1016/j.ijpharm.2015.04.053
WO 2009/109550 A1—Process for Stabilizing an Adjuvant Containing Vaccine Composition
WO 2013/050156 A1—Process Line for The Production of Freeze-Dried Particles
WO 2013/050158 A1—Heating Device for Rotary Drum Freeze-Dryer
WO 2013/050159 A1—A Process for The Bulkware Production of Freeze-Dried Particles Under Closed Conditions
WO 2013/050162 A1—Process Line for The Production of Freeze-Dried Particles
WO 2015/024667A1—Method for Increasing Expression of Rna-Encoded Proteins
WO 2016/012414 A1—Liquid Feeding Device for The Generation of Droplets
WO 2020/139783 A2—Polypeptides Utiles Pour L'edition De Gènes Et Procédés D'utilisation
WO 2022/099003 A1—Lipid Nanoparticles for Delivering mRNA Vaccines
Zhao P, Hou X, Yan J, et al. Long-term storage of lipid-like nanoparticles for mRNA delivery. Bioact Mater. 2020; 5(2):358-363. Published 2020 Mar. 18. doi:10.1016/j.bioactmat.2020.03.001

1. A method for freezing lipid nanoparticles (LNPs), said LNPs comprising at least, as lipid components, a cationic ionizable lipid, a neutral lipid, and a steroid alcohol, or an ester thereof, said LNPs comprising at least a nucleic acid, wherein said method comprises the steps of:
a) providing a liquid composition comprising said LNPs,
b) spraying the composition of step a) in conditions suitable for obtaining liquid droplets, and
c) freezing the liquid droplets obtained at step b) to obtain frozen LNPs.

2. The method according to claim 1, wherein step b) of spraying is carried out with an electromagnetic droplet stream generator, a piezoelectric droplets stream generator, a hydraulic droplets aerosol generator, a pneumatic nozzle, a ultrasonic spray nozzle, a thermal droplets stream generator, or an electrohydrodynamic droplets (EHD) generator.

3. The method according to claim 1, wherein step c) of freezing is carried out by spraying the liquid droplets into a cryogenic atmosphere, with compressed carbon dioxide, into vapor over a cryogenic liquid, into a cryogenic liquid, or onto a cold solid surface.

4. A method for freeze-drying lipid nanoparticles (LNPs), said method comprises at least the steps of:
d) obtaining frozen LNPs according to the method of claim 1, and
e) drying the frozen LNPs obtained at step d) under conditions suitable to obtain freeze-dried LNPs.

5. The method according to claim 4, wherein step e) of drying is carried out by rotary drum vacuum lyophilization, atmospheric drying with a flow of cold air, vacuum chamber lyophilization, or vacuum tunnel lyophilization.

6. The method according to claim 1, wherein the LNPs comprise:
from 20 to 60%, or from 25% to 60%, or from 30% to 55%, or from 35% to 55%, or from 35% to 50%, or from 40% to 50%, of said ionizable cationic lipid, and/or
from 5 to 50%, or from 5% to 45%, or from 9% to 40%, or from 9% to 30%, of said neutral lipid, and/or
from 20 to 55%, or from 20% to 50%, or from 25% to 45%, of said steroid alcohol, or ester thereof,
in % w/w relative to the total weight of the lipid components of said LNPs.

7. The method according to claim 1, wherein:
the ionizable cationic lipid is selected from the group comprising [(6Z,9Z,28Z,31Z)-heptatriaconta-6,9,28,31-tetraen-19-yl] 4-(dimethylamino)butanoate (D-Lin-MC3-DMA); 2,2-dilinoleyl-4-dimethylaminoethyl-[1,3]-dioxolane (DLin-KC2-DMA); 1,2-dilinoleyloxy-N,N-dimethyl-3-aminopropane (DLin-DMA); di((Z)-non-2-en-1-yl) 9-((4-(dimethylamino)butanoyl)oxy)heptadecanedioate (L319); 9-heptadecanyl 8-{(2-hydroxyethyl)[6-oxo-6-(undecyloxy)hexyl]amino}octanoate (SM-102); [(4-hydroxybutyl)azanediyl]di(hexane-6,1-diyl) bis(2-hexyldecanoate) (ALC-0315); [3-(dimethylamino)-2-[(Z)-octadec-9-enoyl]oxypropyl] (Z)-octadec-9-enoate (DODAP); 2,5-bis(3-aminopropylamino)-N-[2-[di(heptadecyl)amino]-2-oxoethyl]pentanamide (DOGS); [(3S,8S,9S,10R,13R,14S,17R)-10,13-dimethyl-17-[(2R)-6-methylheptan-2-yl]-2,3,4,7,8,9,11,12,14,15,16,17-dodecahydro-1H-cyclopenta[a]phenanthren-3-yl] N-[2-(dimethylamino)ethyl]carbamate (DC-Chol); tetrakis(8-methylnonyl) 3,3′,3″,3′″-(((methylazanediyl) bis(propane-3,1 diyl))bis (azanetriyl))tetrapropionate (306Oi10); decyl (2-(dioctylammonio)ethyl) phosphate (9A1P9); ethyl 5,5-di((Z)-heptadec-8-en-1-yl)-1-(3-(pyrrolidin-1-yl)propyl)-2,5-dihydro-1H-imidazole-2-carboxylate (A2-Iso5-2DC18); bis(2-(dodecyldisulfanyl)ethyl) 3,3′-((3-methyl-9-oxo-10-oxa-13,14-dithia-3,6-diazahexacosyl)azanediyl)dipropionate (BAME-O16B); 1,1′-((2-(4-(2-((2-(bis(2-hydroxydodecyl)amino)ethyl) (2-hydroxydodecyl)amino)ethyl) piperazin-1-yl)ethyl)azanediyl) bis(dodecan-2-ol) (C12-200); 3,6-bis(4-(bis(2-hydroxydodecyl)amino)butyl)piperazine-2,5-dione (cKK-E12); hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate (FTT5); (((3,6-dioxopiperazine-2,5-diyl)bis(butane-4, 1-diyl))bis(azanetriyl))tetrakis(ethane-2,1-diyl) (9Z,9′Z,9″Z,9′Z,12Z,12′Z,12″Z,12′Z)-tetrakis (octadeca-9,12-dienoate) (OF-Deg-Lin); TT3; N1,N3,N5-tris(3-(didodecylamino)propyl)benzene-1,3,5-tricarboxamide; N1-[2-((1S)-1-[(3-aminopropyl)amino]-4-[di(3-aminopropyl)amino]butylcarboxamido)ethyl]-3,4-di[oleyloxy]-benzamide (MVL5); heptadecan-9-yl 8-((2-hydroxyethyl)(8-(nonyloxy)-8-oxooctyl)amino)octanoate (Lipid 5); 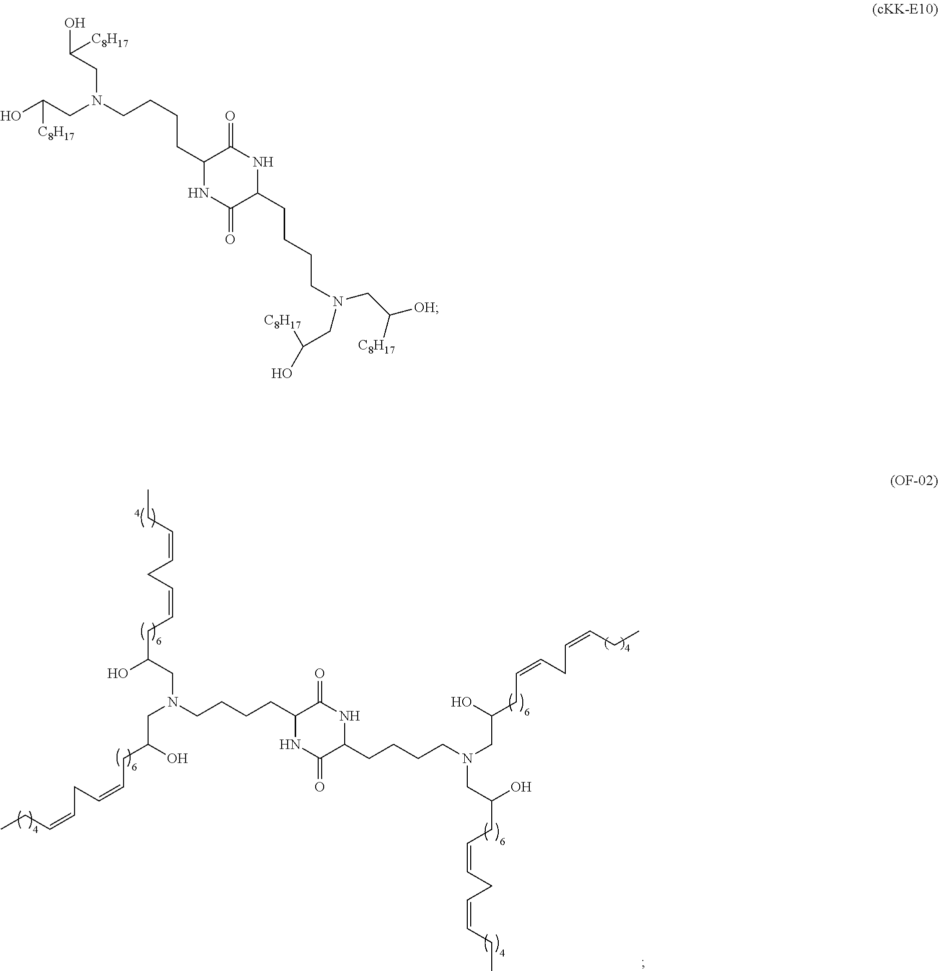
and combinations thereof, and/or
the neutral lipid is selected from the group comprising DSPC; DPPC; DMPC; POPC; DOPC; phosphatidylethanolamines, such as DOPE, DPPE, DMPE, DSPE, DLPE; sphingomyelins; ceramides, and combinations thereof, and/or
the sterol, or an ester thereof, is selected from the group consisting of cholesterol and its derivatives; ergosterol; desmosterol (38-hydroxy-5,24-cholestadiene); stigmasterol (stigmasta-5,22-dien-3-ol); lanosterol (8,24-lanostadien-3b-ol); 7-dehydrocholesterol (Δ5,7-cholesterol); dihydrolanosterol (24,25-dihydrolanosterol); zymosterol (5α-cholesta-8,24-dien-38-ß1); lathosterol (5α-cholest-7-en-3ß-ol); diosgenin ((3β,25R)-spirost-5-en-3-ol); sitosterol (22,23-dihydrostigmasterol); sitostanol; campesterol (campest-5-en-3ß-o1); campestanol (5a-campestan-3b-ol); 24-methylene cholesterol (5,24(28)-cholestadien-24-methylen-3-ß1); cholesteryl margarate (cholest-5-en-3ß-yl heptadecanoate); cholesteryl oleate; cholesteryl stearate; and combinations thereof.

8. The method according to claim 1, wherein the LNPs further comprise as lipid components at least one PEG-lipid.

9. The method according to claim 8, wherein the LNPs comprise from 0.5 to 15%, or from 0.5% to 10%, or from 0.8% to 5%, or from 1% to 3%, or from 1.5% to 2% of said PEG-lipid, in % w/w relative to the total weight of the lipid components of said LNPs.

10. The method according to claim 8, wherein the PEG-lipid is selected from the group consisting of PEG-DAG; DMG-PEG-2000; PEG-PE; PEG-S-DAG; PEG-S-DMG; PEG-cer; a PEG-dialkyoxypropylcarbamate; 2-[(polyethylene glycol)-2000]-N,N-ditetradecylacetamide (ALC-0159); and combinations thereof.

11. The method according to claim 8, wherein the LNPs comprise:
50% of ionizable cationic lipid, 10% of neutral lipid, 38.5% of cholesterol, and 1.5% of PEG-lipid, or
46.3% of ionizable cationic lipid, 9.4% of neutral lipid, 42.7% of cholesterol, and 1.6% of PEG-lipid, or
47.4% of ionizable cationic lipid, 10% of neutral lipid, 40.9% of cholesterol, and 1.7% of PEG-lipid, or
40% of ionizable cationic lipid, 30% of neutral lipid, 28.5% of cholesterol, and 1.5% of PEG-lipid, or
50% of 9-heptadecanyl 8-{(2-hydroxyethyl)[6-oxo-6-(undecyloxy)hexyl]amino}octanoate (SM-102), 10% of DSPC, 38.5% of cholesterol, and 1.5% of DMG-PEG-2000, or
46.3% of [(4-hydroxybutyl)azanediyl]di(hexane-6,1-diyl) bis(2-hexyldecanoate) (ALC-0315), 9.4% of DSPC, 42.7% of cholesterol, and 1.6% of 2-[(polyethylene glycol)-2000]-N,N-ditetradecylacetamide (ALC-0159),
47.4% of [(4-hydroxybutyl)azanediyl]di(hexane-6,1-diyl) bis(2-hexyldecanoate) (ALC-0315), 10% of DSPC, 40.9% of cholesterol, and 1.7% of 2-[(polyethylene glycol)-2000]-N,N-ditetradecylacetamide (ALC-0159), or
40% of cKK-E10, 30% of DOPE, 28.5% of cholesterol, and 1.5% of DMG-PEG-2000, or
40% of OF-02, 30% of DOPE, 28.5% of cholesterol, and 1.5% of DMG-PEG-2000, in % w/w relative to the total weight of the lipid components of said LNPs.

12. The method according to claim 1, wherein the nucleic acid is an RNA.

13. The method according to claim 1, wherein the nucleic acid is an messenger RNA (mRNA); a microRNA (miRNA); a short (or small) interference RNA (siRNA); small hairpin RNA (shRNA); a long non-coding RNA (lncRNA); an asymmetrical interfering RNA (aiRNA); a self-amplifying RNA (saRNA); a guide RNA (gRNA); and combinations thereof.

14. The method according to claim 1, wherein the nucleic acid encodes for a therapeutic agent chosen among a genome-editing polypeptide, a chemokine, a cytokine, a growth factor, an antibody, an enzyme, a structural protein, a blood protein, an hormone, a transcription factor, or an antigen.

15. The method according to claim 1, wherein the liquid composition comprising said LNPs comprise a cryoprotectant.

16. The method according to claim 15, wherein the cryoprotectant is a mixture of trehalose and dextran.

17. The method according to claim 16, wherein the trehalose and the dextran are present in an equal amount of weight by volume percent, relative to the total volume of the composition.

18. Freeze-dried LNPs obtainable according to the method as defined in claim 4.

19. Freeze-dried LNPs comprising at least a nucleic acid and, at least, as lipid components, a cationic ionizable lipid, a neutral lipid, and a steroid alcohol, or an ester thereof, said freeze-dried LNPs being in freeze-dried micropellets.

20. Freeze-dried LNPs according to claim 15, wherein the nucleic acid encodes for a therapeutic agent chosen among a genome-editing polypeptide, a chemokine, a cytokine, a growth factor, an antibody, an enzyme, a structural protein, a blood protein, an hormone, a transcription factor, or an antigen.
